(19) United States
(12) Patent Application Publication
(10) Pub. No.: US 2014/0193802 A1
(43) Pub. Date: Jul. 10, 2014

(54) IDENTIFICATION OF AN ALTERED THERAPEUTIC SUSCEPTIBILITY TO ANTI-HCMV COMPOUNDS AND OF A RESISTANCE AGAINST ANTI-HCMV COMPOUNDS

(71) Applicant: AiCuris GmbH & Co. KG, Wuppertal (DE)

(72) Inventor: Peter LISCHKA, Duesseldorf (DE)

(21) Appl. No.: 13/940,149

(22) Filed: Jul. 11, 2013

Related U.S. Application Data
(60) Provisional application No. 61/849,840, filed on Jul. 11, 2012.

(51) Int. Cl. C12Q 1/70

(52) U.S. Cl. CPC C12Q 1/705
USPC 435/5

(57) ABSTRACT

The present invention relates to a method for the detection of an altered therapeutic response of a subject infected by HCMV to a treatment with a 3,4 dihydroquinazoline or N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide, a method for the detection of a drug resistance of a HCMV to a 3,4-dihydroquinazoline or N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide, and to a method for the detection of a mutation of a HCMV resulting in a drug resistance to a 3,4-dihydroquinazoline or N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide.

### CROSS-REFERENCE TO RELATED APPLICATION

This application claims priority to U.S. Ser. No. 13/546,773, filed Jul. 11, 2012, requested to be converted to a Provisional application. The content of this application is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 595282001120SeqList.txt, date recorded: Jul. 11, 2013, size: 25,197 bytes).

### BACKGROUND OF THE INVENTION

1. Field of the Invention

The present invention relates to a method for the identification of an altered therapeutic susceptibility of a subject infected by a human cytomegalovirus (HCMV) to a treatment with a 3,4-dihydroquinazoline or a compound according to Formula (X):

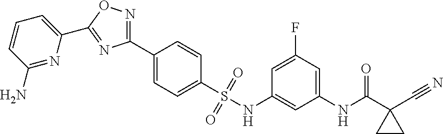 [SMILES: N#CC1(C(=O)Nc2cc(F)cc(NS(=O)(=O)c3ccc(-c4noc(-c5cccc(N)n5)n4)cc3)c2)CC1]

(Formula X); i.e., N-{3-[({4-[5-(6-Aminopyridin-2-yl)-1,2,4-oxadiazol-3-yl]phenyl}sulfonyl)amino]-5-fluorophenyl}-1-cyanocyclopropanecarboxamide,

a method for the identification of a drug resistance of a HCMV to a 3,4-dihydroquinazoline or the compound of Formula (X), and to a method for the identification of a mutation of a HCMV resulting in a drug resistance to a 3,4-dihydroquinazoline or the compound of Formula (X).

2. Related Prior Art

Human cytomegalovirus is a species of virus that belongs to the viral family known as Herpesviridae or herpesviruses. It is typically abbreviated as HCMV and is alternatively known as human herpesvirus-5 (HHV-5). Within Herpesviridae, HCMV belongs to the Betaherpesvirinae subfamily, which also includes cytomegaloviruses from other mammals.

Although they may be found throughout the body, HCMV infections are frequently associated with the salivary glands. HCMV infection is typically unnoticed in healthy people, but can be life-threatening for the immunocompromised, such as HIV-infected persons, organ transplant recipients, or newborn infants. In particular, HCMV remains the leading viral cause of birth defects and life-threatening disease in transplant recipients.

Currently approved anti-HCMV drugs target the viral DNA polymerase, pUL54. Ganciclovir (GCV) acts as nucleoside analogue. Its antiviral activity requires phosphorylation by the HCMV protein kinase, pUL97. Cidovir (CDV) is a nucleotide analogue, which is already phosphorylated and thus active. Foscarnet (FOS) has a different mode of action. It directly inhibits polymerase function by blocking the pyrophosphate binding site of pUL54. Such drugs are associated with severe toxicity issues and the emergence of drug resistance.

An approach to the development of an orally active, less toxic HCMV antiviral with a new mode of action has been the synthesis and evaluation of benzimidazole ribonucleosides. Drugs of this class were shown to be highly active against HCMV and target the viral terminase complex. However, it turned out that such compounds were metabolically unstable. Furthermore, HCM viruses resistant to benzimidazole ribonucleosides have been described where the resistance has been mapped to the viral ORFs UL89 and UL56; cf. Krosky et al., “Resistance of Human Cytomegalovirus to Benzimidazole Ribonucleosides Maps to Two Open Reading Frames: UL89 and UL56,” Journal of Virology, 1998, p. 4721-4728, and Evers et al., “Inhibition of Human Cytomegalovirus Replication by Benzimidazole Nucleosides Involves Three Distinct Mechanisms,” Antimicrobial Agents and Chemotherapy, 2004, p. 3918-3927.

BAY 38-4766 is another potent and selective inhibitor of HCMV replication and a representative of a novel non-nucleosidic class of anti-HCMV-drugs, the phenylenediamine sulfonamides. It also targets the viral terminase complex. BAY 38-4766 prevents the cleavage of high molecular weight viral DNA concatamers to monomeric genomic lengths. However, the development of such compound was discontinued. Furthermore, also for such compound resistant HCM viruses have been described which inter alia contain mutations in the viral ORFs UL56 and UL89; cf. Buerger et al., “A Novel Nonnucleoside Inhibitor Specifically Targets Cytomegalovirus DNA Maturation via the UL89 and UL56 Gene Products,” Journal of Virology, 2001, p. 9077-9086.

Attempts to discover improved anti-HCMV drugs led to the identification of the small-molecular-weight compounds of Formula (X) as depicted above and to 3,4-dihydroquinazolines, such as (S)-{8-Fluoro-2-[4-(3-methoxyphenyl)-1-piperazinyl]-3-[2-methoxy-5-(trifluoromethyl)phenyl]-3,4-dihydro-4-quinazolinyl}acetic acid (Letermovir).

The precise chemical name of Letermovir is (S)-{8-fluoro-2-[4-(3-methoxyphenyl)-1-piperazinyl]-3-[2-methoxy-5-(trifluoromethyl)phenyl]-3,4-dihydro-4-quinazolinyl}acetic acid. The synthesis is disclosed in US 2007/0191387 A1, exemplary embodiments 14 and 15, pages 40 and 41, paragraphs [0495] to [0505]. Letermovir exhibits an outstanding anti-HCMV activity in vitro and in vivo and has completed clinical phase IIb trial. Letermovir inhibits HCMV replication through a specific antiviral mechanism that involves the viral terminase subunit, but that is distinct from that of other compound classes also known to target this enzyme complex; cf. Goldner et al., “The Novel Anticytomegalovirus Compound AIC246 (Letermovir) Inhibits Human Cytomegalovirus Replication through a Specific Antiviral Mechanism That Involves the Viral Terminase,” Journal of Virology, 2011, p. 10884-10893.

The anti-HCMV activities of the compound of Formula (X), which precise chemical name is N-{3-[({4-[5-(6-Aminopyridin-2-yl)-1,2,4-oxadiazol-3-yl]phenyl}sulfonyl)amino]-5-fluorophenyl}-1-cyanocyclopropanecarboxamide and its synthesis are disclosed in US2009/0176842 A1, exemplary embodiment 1, page 15, paragraphs [0298] to [0302].

However, the inventors have recently realized that several HCM viruses selected in cell culture escape inhibition by the compound of Formula (X) and 3,4-dihydroquinazolines, respectively; cf. Goldner et al. (2012; l.c.). The inventors have also realized that the administration of 3,4-dihydroquinazoline or the compound of Formula (X) itself can induce resistances to such substance. Therefore, patients infected with those resistant viruses would exhibit a reduced therapeutic response to a treatment with a 3,4-dihydroquinazoline or the compound of Formula (X).

If such a reduced therapeutic response of a subject infected by HCMV would be detectable before starting a medication or during therapy a better basis for a targeted administration of anti-HCMV compounds would be given.

The person skilled in the art understands that the compound of Formula (X) is mere illustrative for the respective compound class of anti-HCMV agents, namely the phenylenediamine sulfonamides having the characteristic chemical group —SO₂—NHR. Therefore, the subject matter of the present invention also encompasses an in vitro method for the identification of an altered therapeutic susceptibility of a subject infected by HCMV to a method of treatment with an anti-HCMV agent selected from the phenylenediamine sulfonamides having the characteristic chemical group —SO₂—NHR.

### SUMMARY OF THE INVENTION

Therefore, a problem underlying the invention is to provide a method for the identification of an altered therapeutic susceptibility of a subject infected by HCMV to a treatment with a 3,4-dihydroquinazoline or the compound of Formula (X).

Such problem is solved by providing a method comprising the following steps: 1. providing genetic material of the HCMV isolated from said subject, 2. screening said genetic material for at least one mutation in the ORF UL56, and 3. correlating a positive finding in step 2 with the presence of an altered, preferably reduced therapeutic susceptibility.

As regards step 1, the provision of genetic material of the HCMV is not literally restricted to the “isolation” thereof. All methods known to the person skilled in the art to obtain the genetic information of the HCMV of interest are encompassed by the scope of the instant invention.

As regards step 2, said “screening” of the genetic material obtained under step 1 for at least one mutation in the ORF UL56 in accordance with the invention, is not restricted to standard methods of sequencing. Step 2 may be also realized by any method suitable to detect nucleic acid or amino acid mutations (or polymorphisms) within the ORF UL56 of the HCMV genetic material, including but not limited to nucleic acid sequencing technologies, hybridization technologies, DNA chip technologies, melting curve analyses, denaturing high performance liquid chromatography (dHPLC), in accordance with the invention. Therefore, the term “screening” encompasses nucleic acid sequencing technologies, hybridization technologies, DNA chip technologies, melting curve analyses, denaturing high performance liquid chromatography (dHPLC), or any other method that is suitable to detect nucleic acid or amino acid mutations or polymorphisms within HCMV genetic material, particularly within the ORF UL56 thereof.

In this regard, the person skilled in the art understands that the method used for either obtaining the HCMV genetic material or screening said material for at least one mutation in the ORF UL56 does not influence the correlation step under step 3 of the instant invention. Thus, any method suitable for obtaining the HCMV genetic material and for screening said material for at least one mutation in the ORF UL56 enables the person skilled in the art to put the present invention into practice, which means to provide for therapy guidance in view of the administration of a 3,4-dihydroquinazoline or the compound of Formula (X) as anti-HCMV agent.

The person skilled in the art further understands that not a particular mutation in the ORF UL56 has to be present, but rather any of the point mutations found by the inventors enables the person skilled in the art to correlate a positive or negative finding with the presence or absence of an altered, preferably reduced therapeutic susceptibility of a subject infected by HCMV to a method of treatment with a 3,4-dihydroquinazoline or the compound of Formula (X).

Another problem underlying the invention is to provide a method for the identification of a drug resistance of a HCMV to a 3,4-dihydroquinazoline or the compound of Formula (X).

Such problem is solved by providing a method comprising the following steps: 1. providing genetic material of the HCMV, 2. screening said genetic material for at least one mutation in the ORF UL56, and 3. correlating a positive finding in step 2 with the presence of a drug resistance.

Another problem underlying the invention is to provide a method for the identification of a mutation of a HCMV resulting in a drug resistance to a 3,4-dihydroquinazoline or the compound of Formula (X).

Such problem is solved by providing a method comprising the following steps: 1. providing genetic material of the HCMV, and 2. screening said genetic material for at least one mutation in the ORF UL56.

These solutions are surprising and could not be foreseen.

Even though the viral ORF56 has been described in connection with an HCMV being resistant to BAY 38-4766 and to benzimidazole ribonucleosides, the group of 3,4-dihydroquinazolines are chemically distinctive substances which act via a mode of interaction with the viral terminase that is distinct from that of these compounds.

BAY 38-4766 belongs to the group of phenylenediamine sulfonamides and targets the viral terminase complex. Benzimidazole ribonucleosides, such as BDCRB and maribavir, are chemically and functionally distinct. BDCRB block the processing and maturation of viral DNA by targeting the viral terminase. Maribavir prevents the viral DNA synthesis and capsid nuclear egress.

To the contrary, 3,4-dihydroquinazoline, such as Letermovir, block the viral replication without inhibiting the synthesis of progeny HCMV DNA or viral proteins. In fact, Letermovir, respectively, was also shown to act via a mode of action that involves the viral terminase. However, its mode of interaction with the viral terminase complex and its chemical structure is distinct from that of all other thus-far characterized drugs known to target the HCMV terminase complex, including BDCRB and BAY 38-4766. While an antiviral activity against rodent cytomegaloviruses was described for all published cleavage/packaging inhibitors, including said BDCRB and BAY38-4766, Letermovir is solely active against the human cytomegalovirus. See hereto Marschall, M., T. Stamminger, A. Urban, S. Wildum, H. Ruebsamen-Schaeff, H. Zimmermann, and P. Lischka, “In Vitro Evaluation of the Activities of the Novel Anticytomegalovirus Compound AIC246 (Letermovir) against Herpesviruses and Other Human Pathogenic Viruses,” Antimicrob. Agents Chemother (2012) 56:1135-1137.

The compound of Formula (X) belongs to the class of phenylenediamine sulfonamides and targets the viral terminase complex.

Therefore, it could not have been expected that mutations in the viral ORF56 are responsible for conferring resistance of the HCMV to a 3,4-dihydroquinazoline or the compound of Formula (X).

According to the invention a “subject” refers to any living being, in particular a mammal such as a human being.

According to the invention “identification” refers to any measure, which aims for a search for an amended, preferably reduced therapeutic susceptibility, drug resistance or drug-resistance conferring mutation. It includes a prediction before, or detection during or after an anti-HCMV medication.

According to the invention, “altered or reduced therapeutic susceptibility” refers to a response of a subject to a 3,4-dihydroquinazoline or the compound of Formula (X) which is, in comparison to the response of a reference subject infected by an HCMV being non-resistant to 3,4-dihydroquinazoline or the compound of Formula (X) or wild-type HCMV, respectively, altered or diminished in its size, including a non-response.

According to the invention “at least one mutation” means that the genetic material of HCMV can be screened for more than one mutation, i.e., two, three, four, five, six, seven, eight, nine, ten or more. In particular, the inventors have indications that resistant HCM viruses exist which carry more than one mutation, e.g., L241P and R369S, resulting in an increased resistance.

According to a further development of the methods according to the invention, said 3,4-dihydroquinazoline is Letermovir.

This measure has the advantage that the method is adapted to evaluate the susceptibility of a HCMV infected subject to one of the currently most promising anti-HCMV compounds. Letermovir, respectively, is extensively described in Lischka, P. et al., “In Vitro and In Vivo Activities of the Novel Anticytomegalovirus Compound AIC246.” Antimicrob. Agents Chemother. 2010, 54: p. 1290-1297, and Kaul et al., “First report of successful treatment of multidrug-resistant cytomegalovirus disease with the novel anti-CMV compound AIC246.” Am. J Transplant. 2011, 11:1079-1084; Marschall et al., “In Vitro Evaluation of the Activities of the Novel Anticytomegalovirus Compound AIC246 (Letermovir) against Herpesviruses and Other Human Pathogenic Viruses.” Antimicrob. Agents Chemother, 2012, 56:1135-1137. See hereto Goldner, T., G. Hewlett, N. Ettischer, H. Ruebsamen-Schaeff, H. Zimmermann, and P. Lischka, 2011. “The Novel Anticytomegalovirus Compound AIC246 (Letermovir) Inhibits Human Cytomegalovirus Replication through a Specific Antiviral Mechanism That Involves the Viral Terminase.” J Virol. 85:10884-10893 and Chemaly R F, Ehninger G, Champlin R, Richard M P, Zimmermann H, Lischka P, Stoelben S, McCormick D, Ruebsamen-Schaeff H. “Letermovir (AIC246) for the prevention of CMV infections meets primary endpoint in Phase 2b trial in human blood precursor cell transplant (HBPCT) recipients.” Abstracts of the 52nd Intersci. Conf. Antimicrob Agents Chemother (ICAAC), San Francisco, Calif. 2012 as well.

According to a further development of the methods according to the invention, said genetic material provided in step (1) is viral DNA.

This measure has the advantage that a basis is established for the subsequent screening of the genetic material.

According to a further development of the methods according to the invention, in step (2) said genetic material is screened for such at least one mutation being located in an ORF UL56 section encoding amino acids at positions 200 to 400, preferably 230 to 370, numbered in accordance with the UL56 protein of wild type HCMV (“Merlin”).

According to another development of the methods according to the invention, in step (2) said genetic material is screened for such at least one mutation being located in an ORF UL56 section encoding amino acids at positions 231 to 369, numbered in accordance with the UL56 protein of wild type HCMV (“Merlin”).

The inventors have realized that a hot-spot region can be identified in the UL56 protein of wild type HCMV. Such region which is responsible for resistances to 3,4-dihydroquinazoline such as Letermovir is located between amino acids at positions 200 to 400, or preferably 230 to 370, respectively, where the inventors were able to identify at least 8 point mutations. The indicated positions refer to the UL56 protein of the wild type HCMV designated as “Merlin”. The amino acid sequence of the UL56 protein of “Merlin” can be found under the NCBI protein ID YP_{—}081515.1 and in the attached sequence listing under SEQ ID NO:1. The amino acid sequence of the UL56 protein of the laboratory HCMV strain AD169 is identical regarding the amino acids at positions 200 to 400 and can be found under the NCBI protein YP_{—}002608261.1 and in the attached sequence listing under SEQ ID NO:2. The entire genome of the wild type HCMV “Merlin” can be found under the NCBI accession No. NC_{—}006273.2. The entire genome of the HCMV stem AD169 can be found under the NCBI accession No. NC_{—}001347.6.

The inventors also have realized that in the UL56 protein of wild type HCMV a region which is responsible for resistances to 3,4-dihydroquinazoline such as Letermovir is located between amino acids at positions 231 to 369, where the inventors were able to identify at least 8 point mutations.

Whereas the amino acid sequence of the UL56 protein is well conserved among the known HCMV stems the UL56 coding or nucleotide sequence varies, in particular due to the degeneration of the genetic code. The nucleotide sequence of the UL56 ORF of wild type HCMV “Merlin” can be retrieved under the NCBI gene ID 3077457 (SEQ ID NO:3) and of the HCMV AD169 under the NCBI gene ID 1487751. It goes without saying that all UL56 coding sequences are encompassed by the invention which, after being translated result in a UL56 derived peptide comprising amino acids at positions 200 to 400 or 230 to 370, respectively.

According to a preferred embodiment in step (2) said genetic material is screened for such at least one mutation being located in an ORF UL56 section comprising nucleotides at positions 598 to 1200, preferably 691 to 1107, numbered in accordance with the UL56 ORF of HCMV AD169.

The indicated nucleotide positions frame the hot-spot which is responsible for resistances to 3,4-dihydroquinazoline such as Letermovir and the section of the ORF UL56 which encodes the amino acids at positions 200 to 400, or 230 to 370, respectively.

The indicated nucleotide positions frame the hot-spot which is responsible for resistances to 3,4-dihydroquinazoline such as Letermovir and the section of the ORF UL56 which encodes the amino acids at positions 231 to 369.

According to a preferred development of the methods according to the invention, in step (2) said genetic material is screened for such at least one mutation in the ORF UL56 resulting in an amino acid substitution at a position, numbered in accordance with the UL56 protein of wild type HCMV (“Merlin”), which is selected from the group consisting of: 231, 232, 236, 241, 325, 369.

By this measure the method is directed to a search for such positions in the UL56 protein where the inventors have been able to identify point mutations conferring resistance to 3,4-dihydroquinazoline such as Letermovir. The accuracy and reliability of the method according to the invention is thereby further increased. Interestingly, even though resistance-conferring mutations in the UL56 protein have been described for BAY38-4766 or BDCRB, such mutations are located at different positions in the UL56 protein. Therefore, the UL56 mutations conferring Letermovir resistance seem to be highly specific and emphasize the chemical and functional difference of that substance from currently approved anti-HCMV drugs.

The person skilled in the art understands that the methods disclosed herein for the use in the identification of an altered therapeutic susceptibility of a subject infected by a human cytomegalovirus (HCMV) to a treatment with a 3,4-dihydroquinazoline or a compound according to Formula (X) are in vitro methods.

According to further preferred embodiments said position 231 is substituted with a leucine (V231L), said position 232 is substituted with an aspartic acid (N232D), said position 236 is substituted with a methionine (V236M), said position 241 is substituted with a proline (L241P), said position 325 is substituted with a tyrosine (C325Y), and said position 369 is substituted with serine (R369S), methionine (R369M), and/or glycine (R369G).

This measure has the advantage that the provided genetic material is screened for such specific point mutations conferring resistance to a 3,4-dihydroquinazoline such as Letermovir the inventors were able to identify in the UL56 ORF of various HCMV isolates.

The inventors were also able to identify a hot spot in the UL56 ORF that confers resistance to the compound of Formula (X), namely in an section encoding amino acids at positions 340 to 700, preferably 369 to 662, numbered in accordance with the UL56 protein of wild type HCMV (“Merlin”). Therefore, according to an alternative embodiment of the methods according to the invention, in step (2) said genetic material is screened for such at least one mutation being located in an ORF UL56 section encoding amino acids at positions 340 to 700, preferably 369 to 662 numbered in accordance with the UL56 protein of wild type HCMV (“Merlin”).

According to such alternative embodiment in step (2) said genetic material is screened for such at least one mutation being located in an ORF UL56 section comprising nucleotides at positions 1018 to 2100, preferably 1105 to 1986, numbered in accordance with the UL56 ORF of HCMV AD169. The indicated nucleotide positions frame the hot-spot responsible for resistances to the compound of Formula (X) and the section of the ORF UL56 which encodes the amino acids at positions 340 to 700, or 369 to 662, respectively.

According to such alternative embodiment of the methods according to the invention, in step (2) said genetic material is screened for such at least one mutation in the ORF UL56 resulting in an amino acid substitution at a position, numbered in accordance with the UL56 protein of wild type HCMV (“Merlin”), which is selected from the group consisting of: 369, 407, 447, 529, and 662.

By this measure the alternative methods are directed to a search for such positions in the UL56 protein where the inventors have been able to identify point mutations in HCMV conferring resistance to the compound of Formula (X).

According to a preferred embodiment of the method according to the invention step (1) is preceded by the following step: 1′. isolating genetic material of the HCMV from the subject.

The HCMV can be isolated from various biological material, preferably whole blood, blood plasma or tissue biopsies.

The features and embodiments explained above apply not only to the method for the detection of a reduced therapeutic response of a subject infected by HCMV to a treatment with a 3,4-dihydroquinazoline or the compound of Formula (X), but also to the method for the detection of a drug resistance of a HCMV to a 3,4-dihydroquinazoline or the compound of Formula (X), and the method for the detection of a mutation of a HCMV resulting in a drug resistance to a 3,4-dihydroquinazoline or the compound of Formula (X) correspondingly.

It will be understood that the features of the invention mentioned above and those yet to be explained below can be used not only in the respective combination indicated, but also in other combinations or in isolation, without leaving the scope of the present invention.

Throughout the description the term “3,4-dihydroquinazoline(s)” denote(s) a chemical entity(ies) that is/are generically characterized by the backbone according to Formula (Y):

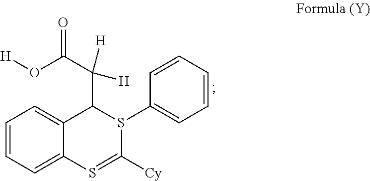

wherein
Cy is selected from a carbocyclic or heterocyclic ring system;

wherein a carbocyclic ring system denotes a saturated hydrocarbon ring group having 5 to 6 ring carbon atoms; a 5- to 6-membered carbocyclic aromatic ring; bicyclic ring systems wherein at least one ring is carbocyclic and aromatic; tricyclic ring systems wherein at least one ring is carbocyclic and aromatic;

and wherein a heterocyclic ring system denotes a 5- to 6-membered aliphatic ring as well as a 5- to 6-membered aromatic ring containing one or more heteroatoms chosen from N, O, and S, with the remaining ring atoms being carbon; and

bicyclic heterocycloalkyl rings containing one or more heteroatoms chosen from N, O, and S, with the remaining ring atoms being carbon and wherein at least one heteroatom is present in an aromatic ring;

and wherein all ring systems, except for the central ring having two nitrogen atoms according to Formula (Y), may be further substituted with one or more heteroatoms chosen from N, O, and S.

Throughout the description the term “3,4-dihydroquinazoline(s)” preferably denote(s) a chemical entity(ies) that is/are generically characterized by the backbone according to any of the Formulae (Y1; Y2; Y3; Y4; Y5):

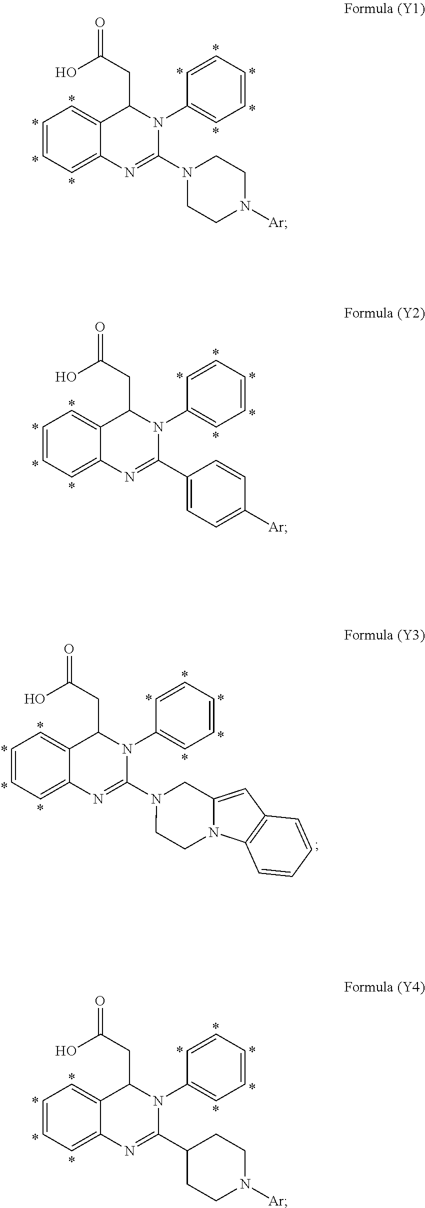

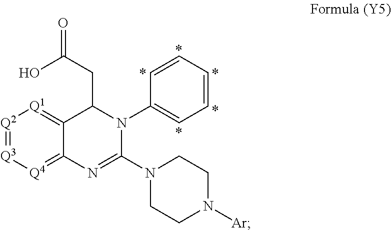

wherein

in any of the Formulae (Y1; Y2; Y3; Y4; Y5) asterisk (*) illustrates potential positions for further substituents; and Ar indicates a further substituent aryl, wherein said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;

and wherein

in Formula (Y5) one or two of Q¹, Q², Q³ and Q⁴ represent N and the others simultaneously represent CH.

The person skilled in the art understands that the Formulae (Y1; Y2; Y3; Y4; Y5) are mere illustrative for the generic compounds according to Formula (Y), and thus the term “3,4-dihydroquinazoline(s)” preferably encompasses the definitions of the compounds of Formula (Y) in accordance with the invention.

Throughout the description the term “3,4-dihydroquinazoline(s)” more preferably denote(s) a chemical entity(ies) having the Formula (Z) and the salts, solvates, or solvates of the salts thereof:

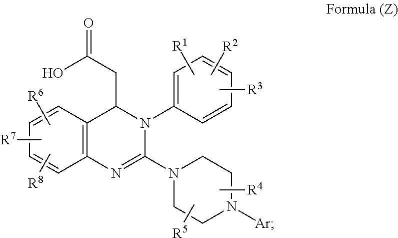

wherein

R¹ is selected from hydrogen, amino, alkyl, alkoxy, alkyl amino, alkyl thio, cyano, halo, nitro, trifluoromethyl, or trifluoromethoxy;

R² is selected from hydrogen, alkyl, alkoxy, alkyl thio, cyano, halo, nitro, or trifluoromethyl;

R³ is selected from amino, alkyl, alkoxy, alkyl amino, alkyl thio, cyano, halo, nitro, trifluoromethyl, alkyl sulfonyl, or alkyl amino sulfonyl;

or one of R¹, R², R³ is selected from hydrogen, alkyl, alkoxy, cyano, halo, nitro, or trifluoromethyl and the both remaining substituents form a 1,3 dioxolane, or a cyclopentane ring, or a cyclohexane ring via its carbon atoms that are bound to the ring system;

R⁴, R⁵ are independently chosen from hydrogen, alkyl, alkoxy or halo;

or R⁴, R⁵ of the piperazine ring are bound to opposite carbon atoms and form a methylene bridge via 1 or 2 methyl groups;

Ar is aryl, and said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;

wherein the alkyl may be further substituted with 1 to 3 substituents independently selected from the group consisting of halo, amino, alkyl amino, hydroxy, and aryl, or two of the substituents form together with the carbon atoms they are bound to a 1,3-dioxolane, or a cyclopentane ring, or a cyclohexane ring, and the third remaining substituent may be independently chosen from the substituents described above for alkyl;

R⁶, R⁷, R⁸ are independently chosen from hydrogen, alkyl, alkoxy, alkyl thio, formyl, carboxyl, alkyl carbonyl, alkoxy carbonyl, amino carbonyl, trifluoromethyl, halo, cyano, hydroxy, or nitro.

Throughout the description the term “3,4-dihydroquinazoline(s)” even more preferably denote(s) a chemical entity(ies) having the Formula (Z) and the salts, solvates, or the salts of the solvates thereof:

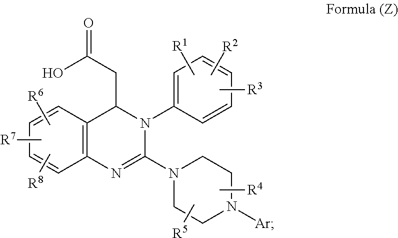

wherein

R¹ is selected from hydrogen, methyl, methoxy, methyl thio, fluoro, or chloro;

R² is selected from hydrogen;

R³ is selected from methyl, tert-butyl, cyano, fluorine, chlorine, nitro, or trifluoromethyl;

R⁴, R⁵ are chosen from hydrogen, or alkyl, or halo;

Ar is phenyl, and wherein said phenyl may be further substituted with 1 or 2 substituents independently selected from the group consisting of methyl, methoxy, fluorine, and chlorine,

R⁶ is hydrogen or fluorine,

R⁷ is hydrogen,

R⁸ is amino carbonyl or fluorine.

Throughout the description the term “3,4-dihydroquinazoline(s)” most preferred denote(s) a chemical entity having the Formula (Z1) and the salts, solvates, or the salts of the solvates thereof:

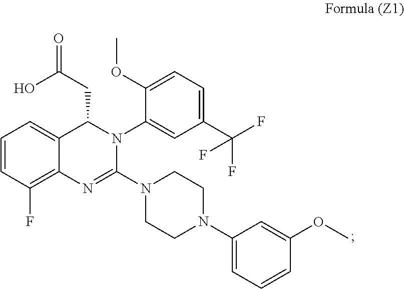 [SMILES: COc1cccc(N2CCN(C3=Nc4c(F)cccc4[C@H](CC(=O)O)N3c3cc(C(F)(F)F)ccc3OC)CC2)c1]

### i.e., Letermovir.

Unless otherwise specified herein, throughout the description the substituents have the following meaning:

“Alkyl” itself as well as “alk” and “alkyl” in alkoxy, alkyl amino, alkyl carbonyl, alkyl sulfonyl, alkyl amino sulfonyl and alkoxy carbonyl denote a branched or unbranched alkyl substituent with 1 to 6, preferably 1 to 4, more preferably 1 to 3 carbon atoms; thus “alkyl” for instance denotes methyl, ethyl, n-propyl, isopropyl, tert-butyl, n-pentyl, and n-hexyl.

“Alkoxy” for instance and preferably denotes methoxy, ethoxy, n-propoxy, isopropoxy, tert-butoxy, n-pentoxy, and n-hexoxy.

“Alkylamino” denotes an alkyl amino substituent having independently one or two alkyl substituents such as methyl amino, ethyl amino, n-propyl amino isopropyl amino, tert-butyl amino, n-pentyl amino, n-hexyl amino, N,N-dimethyl amino, N,N-diethyl amino, N-ethyl-N-methyl amino, N-methyl-N-n-propyl amino, N-isopropyl-N-n-propyl amino, N-tert-butyl-N-methyl amino, N-ethyl-N-n-pentylamino and N-n-hexyl-N-methyl amino. C₁-C₃-alkyl amino for instance denotes a mono-alkyl amino residue with 1 to 3 carbon atoms or a di-alkyl amino residue with 1 to 3 carbon atoms per alkyl substituent.

“Alkyl sulfonyl” for instance and preferably denotes methyl sulfonyl, ethyl sulfonyl, n-propyl sulfonyl, isopropyl sulfonyl, tert-butylsulfonyl, n-pentyl sulfonyl and n-hexyl sulfonyl.

“Alkyl amino sulfonyl” denotes an alkyl amino sulfonyl substituent having independently one or two alkyl substituents such as methyl amino sulfonyl, ethyl amino sulfonyl, n-propyl amino sulfonyl, isopropyl amino sulfonyl, tert-butyl amino sulfonyl, n-pentyl amino sulfonyl, n-hexyl amino sulfonyl, N,N-dimethyl amino sulfonyl, N,N-diethyl amino sulfonyl, N-ethyl-N-methyl amino sulfonyl, N-methyl-N-n-propyl amino sulfonyl, N-isopropyl-N-n-propyl amino sulfonyl, N-tert-butyl-N-methyl amino sulfonyl, N-ethyl-N-n-pentylamino sulfonyl and N-n-hexyl-N-methyl amino sulfonyl. C₁-C₃-alkyl amino sulfonyl for instance denotes a mono-alkyl amino sulfonyl residue with 1 to 3 carbon atoms or a di-alkyl amino sulfonyl residue with 1 to 3 carbon atoms per alkyl substituent.

“Alkyl carbonyl” for instance and preferably denotes acetyl and propanoyl.

“Alkoxy carbonyl” for instance and preferably denotes methoxy carbonyl, ethoxy carbonyl, n-propoxy carbonyl, isopropoxy carbonyl, tert-butoxy carbonyl, n-pentoxy carbonyl, and n-hexoxy carbonyl.

“Aryl” denotes a mono- or tricyclic aromatic, carbocyclic substituent with 6 to 14 carbon atoms; “aryl” for instance denotes phenyl, naphthyl and phenanthrenyl.

“Halogen or halo” denotes fluorine, chlorine, bromine, iodine, preferably fluorine or chlorine.

Further preferred embodiments of the invention are represented by the below consecutively numbered sentences 1a to 42a:
1a. Method for the identification of an altered therapeutic susceptibility of a subject infected by HCMV to a treatment with a 3,4-dihydroquinazoline or a compound of Formula (X)

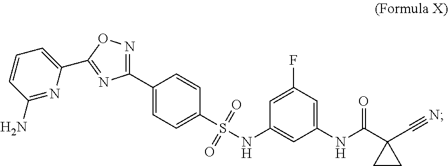 [SMILES: N#CC1(C(=O)Nc2cc(F)cc(NS(=O)(=O)c3ccc(-c4noc(-c5cccc(N)n5)n4)cc3)c2)CC1]

i.e. N-{3-[({4-[5-(6-Aminopyridin-2-yl)-1,2,4-oxadiazol-3-yl]phenyl}sulfonyl)amino]-5-fluorophenyl}-1-cyanocyclopropanecarboxamide, comprising the following steps:
(1) providing genetic material of the HCMV isolated from said subject,
(2) screening said genetic material for at least one mutation in the ORF UL56, and
(3) correlating a positive finding in step (2) with the presence of a reduced therapeutic susceptibility.
2a. Method of sentence 1a, wherein the altered therapeutic susceptibility is a reduced therapeutic susceptibility.
3a. Method of sentence 1a, wherein said 3,4-dihydroquinazoline is Letermovir.
4a. Method of sentence 1a, wherein said genetic material provided in step (1) is viral DNA.
5a. Method of sentence 1a, wherein in step (2) said genetic material is screened for such at least one mutation being located in a ORF UL56 section encoding amino acids at positions 200 to 400 numbered in accordance with the UL56 protein of wild type HCMV (“Merlin”).
6a. Method of sentence 1a, wherein in step (2) said genetic material is screened for such at least one mutation being located in a ORF UL56 section encoding amino acids at positions 230 to 370 numbered in accordance with the UL56 protein of wild type HCMV (“Merlin”).
7a. Method of sentence 1a, wherein in step (2) said genetic material is screened for such at least one mutation being located in a ORF UL56 section comprising nucleotides at positions 598 to 1200 numbered in accordance with the UL56 ORF of HCMV AD169.
8a. Method of sentence 1a, wherein in step (2) said genetic material is screened for such at least one mutation being located in a ORF UL56 section comprising nucleotides at positions 691 to 1107 numbered in accordance with the UL56 ORF of HCMV AD169.
9a. Method of sentence 1a, wherein in step (2) said genetic material is screened for such at least one mutation in the ORF UL56 resulting in an amino acid substitution at a position, numbered in accordance with the UL56 protein of wild type HCMV (Merlin), which is selected from the group consisting of: 231, 232, 236, 241, 325, 369.
10a. Method of sentence 9a, wherein said position 231 is substituted with a leucine (V231L).
11a. Method of sentence 9a, wherein said position 232 is substituted with an aspartic acid (N232D).
12a. Method of sentence 9a, wherein said position 236 is substituted with a methionine (V236M).
13a. Method of sentence 9a, wherein said position 241 is substituted with a proline (L241P).
14a. Method of sentence 9a, wherein said position 325 is substituted with a tyrosine (C325Y).
15a. Method of sentence 9a, wherein said position 369 is substituted with an amino acid selected from the group consisting of: serine (R369S), methionine (R369M), and glycine (R369G).
16a. Method of sentence 9a, wherein step (2) is realized by means of sequencing the ORF UL56 of the genetic material.
17a. Method of sentence 1a, wherein step (1) is preceded by the following step:
1′. isolating genetic material of the HCMV from the subject.
18a. Method of the sentences 1a and 4a to 17a, or of sentence 2a, wherein said 3,4-dihydroquinazoline is a compound having a backbone according to Formula (Y):

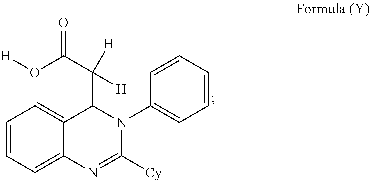

wherein

Cy is selected from a carbocyclic or heterocyclic ring system;

wherein a carbocyclic ring system denotes a saturated hydrocarbon ring group having 5 to 6 ring carbon atoms; a 5- to 6-membered carbocyclic aromatic ring; bicyclic ring systems wherein at least one ring is carbocyclic and aromatic, tricyclic ring systems wherein at least one ring is carbocyclic and aromatic;

wherein a heterocyclic ring system denotes a 5- to 6-membered aliphatic ring as well as a 5- to 6-membered aromatic ring containing one or more heteroatoms chosen from N, O, and S, with the remaining ring atoms being carbon; and

bicyclic heterocycloalkyl rings containing one or more heteroatoms chosen from N, O, and S, with the remaining ring atoms being carbon and wherein at least one heteroatom is present in an aromatic ring;

and wherein all ring systems, except for the central ring having two nitrogen atoms according to Formula (Y), may be further substituted with one or more heteroatoms chosen from N, O, and S.
19a. Method of the sentences 1a and 4a to 18a, or of sentence 2a, wherein said 3,4-dihydroquinazoline is a compound according to any of the Formulae (Y1; Y2; Y3; Y4; Y5):

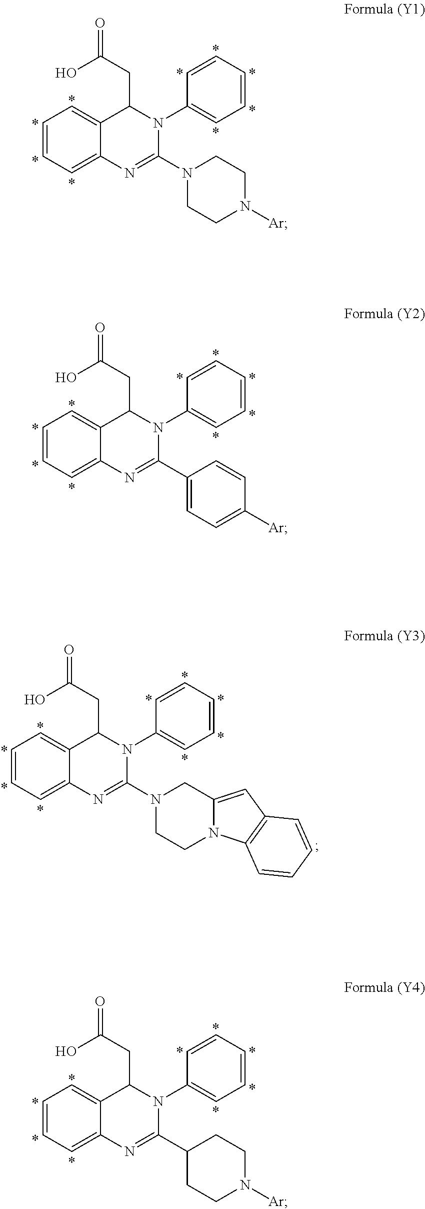

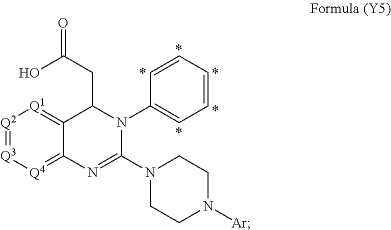

wherein

in any of the Formulae (Y1; Y2; Y3; Y4; Y5) asterisk (*) illustrates potential positions for further substituents; and Ar indicates a further substituent aryl, wherein said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;

and wherein

in Formula (Y5) one or two of Q¹, Q², Q³ and Q⁴ represent N and the others simultaneously represent CH.
20a. Method of the sentences 1a and 4a to 17a, or of sentence 2a, wherein said 3,4-dihydroquinazoline is a compound according to Formula (Z) or a pharmaceutically acceptable salt or solvate thereof or the salt of said solvate thereof:

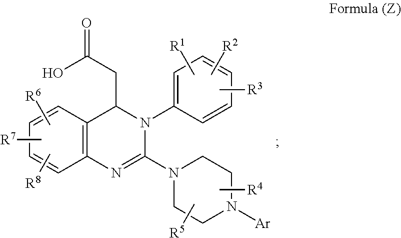

wherein

R¹ is selected from hydrogen, amino, alkyl, alkoxy, alkyl amino, alkyl thio, cyano, halo, nitro, trifluoromethyl, or trifluoromethoxy;

R² is selected from hydrogen, alkyl, alkoxy, alkyl thio, cyano, halo, nitro, or trifluoromethyl;

R³ is selected from amino, alkyl, alkoxy, alkyl amino, alkyl thio, cyano, halo, nitro, trifluoromethyl, alkylsulfonyl, or alkylaminosulfonyl;

or one of R¹, R², R³ is selected from hydrogen, alkyl, alkoxy, cyano, halo, nitro, or trifluoromethyl, and the both remaining substituents form a 1,3 dioxolane, or a cyclopentane ring, or a cyclohexane ring via its carbon atoms that are bound to the ring system;

R⁴, R⁵ are independently chosen from hydrogen, alkyl, alkoxy or halo;

or R⁴, R⁵ of the piperazine ring according to Formula (Z) are bound to opposite carbon atoms and form a methylene bridge via 1 or 2 methyl groups;

Ar is aryl, and said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;

wherein the alkyl may be further substituted with 1 to 3 substituents independently selected from the group consisting of halo, amino, alkyl amino, hydroxy, and aryl,

or two of the substituents form together with the carbon atoms they are bound to a 1,3-dioxolane, or a cyclopentane ring, or a cyclohexane ring, and the third remaining substituent may be independently chosen from the substituents described above for alkyl;

R⁶, R⁷, R⁸ are independently chosen from hydrogen, alkyl, alkoxy, alkyl thio, formyl, carboxyl, alkyl carbonyl, alkoxy carbonyl, amino carbonyl, trifluoromethyl, halo, cyano, hydroxy, or nitro.
21a. Method of the sentences 1a and 4a to 17a and sentence 20a or of sentence 2a, wherein said 3,4-dihydroquinazoline is a compound according to Formula (Z1) or a pharmaceutically acceptable salt or solvate thereof or the salt of said solvate thereof:

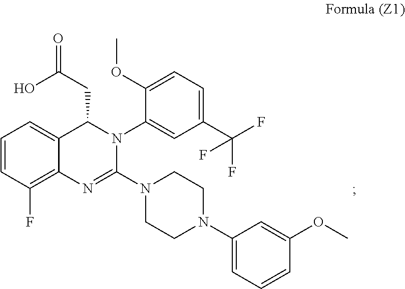 [SMILES: COc1cccc(N2CCN(C3=Nc4c(F)cccc4[C@H](CC(=O)O)N3c3cc(C(F)(F)F)ccc3OC)CC2)c1]

### i.e., Letermovir.

22a. Method of any of the sentences 1a to 21a, wherein said method is an in vitro method.
23a. An in vitro method for use in the identification of an altered therapeutic susceptibility of a subject infected by HCMV to a 3,4-dihydroquinazoline or the compound of Formula (X)

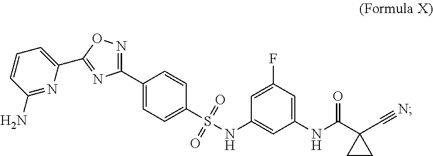 [SMILES: N#CC1(C(=O)Nc2cc(F)cc(NS(=O)(=O)c3ccc(-c4noc(-c5cccc(N)n5)n4)cc3)c2)CC1]

i.e., N-{3-[({4-[5-(6-Aminopyridin-2-yl)-1,2,4-oxadiazol-3-yl]phenyl}sulfonyl)amino]-5-fluorophenyl}-1-cyanocyclopropanecarboxamide, comprising the following steps:
(1) providing genetic material of the HCMV from a subject infected with HCMV,
(2) screening said genetic material for at least one mutation in the ORF UL56, and
(3) correlating a positive finding in step (2) with a reduced therapeutic susceptibility.
24a. The in vitro method of sentence 23a, wherein said 3,4-dihydroquinazoline is a compound having a backbone according to Formula (Y):

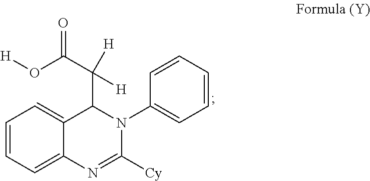

wherein

Cy is selected from a carbocyclic or heterocyclic ring system;

wherein a carbocyclic ring system denotes a saturated hydrocarbon ring group having 5 to 6 ring carbon atoms; a 5- to 6-membered carbocyclic aromatic ring; bicyclic ring systems wherein at least one ring is carbocyclic and aromatic, tricyclic ring systems wherein at least one ring is carbocyclic and aromatic;

wherein a heterocyclic ring system denotes a 5- to 6-membered aliphatic ring as well as a 5- to 6-membered aromatic ring containing one or more heteroatoms chosen from N, O, and S, with the remaining ring atoms being carbon; and

bicyclic heterocycloalkyl rings containing one or more heteroatoms chosen from N, O, and S, with the remaining ring atoms being carbon and wherein at least one heteroatom is present in an aromatic ring,

and wherein all ring systems, except for the central ring having two nitrogen atoms according to Formula (Y), may be further substituted with one or more heteroatoms chosen from N, O, and S.
25a. The in vitro method of the sentences 23a to 24a, wherein said 3,4-dihydroquinazoline is a compound according to any of the Formulae (Y1; Y2; Y3; Y4; Y5):

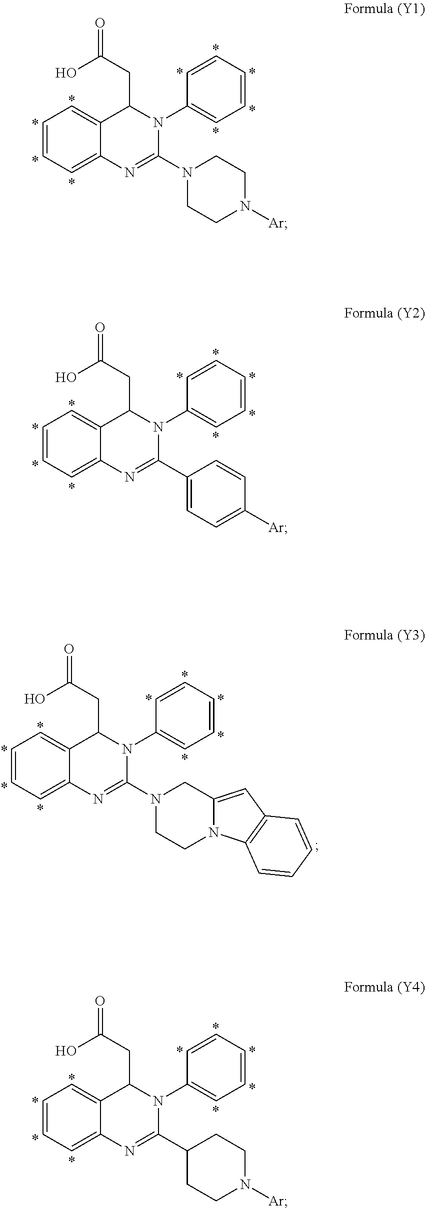

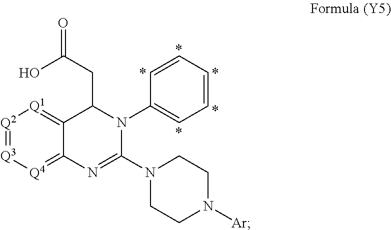

wherein

in any of the Formulae (Y1; Y2; Y3; Y4; Y5) asterisk (*) illustrates potential positions for further substituents; and Ar indicates a further substituent aryl, wherein said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;

and wherein

in Formula (Y5) one or two of Q¹, Q², Q³ and Q⁴ represent N and the others simultaneously represent CH.
26a. The in vitro method of the sentence 23a, wherein said 3,4-dihydroquinazoline is a compound according to Formula (Z):

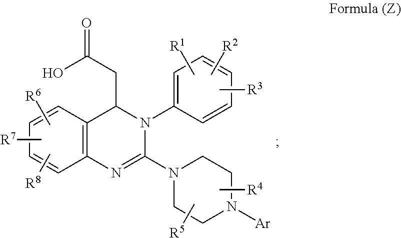

wherein

R¹ is selected from hydrogen, amino, alkyl, alkoxy, alkyl amino, alkyl thio, cyano, halo, nitro, trifluoromethyl, or trifluoromethoxy;

R² is selected from hydrogen, alkyl, alkoxy, alkyl thio, cyano, halo, nitro, or trifluoromethyl;

R³ is selected from amino, alkyl, alkoxy, alkyl amino, alkyl thio, cyano, halo, nitro, trifluoromethyl, alkylsulfonyl, or alkylaminosulfonyl;

or one of R¹, R², R³ is selected from hydrogen, alkyl, alkoxy, cyano, halo, nitro, or trifluoromethyl and the both remaining substituents form a 1,3 dioxolane, or a cyclopentane ring, or a cyclohexane ring via its carbon atoms that are bound to the ring system;

R⁴, R⁵ are independently chosen from hydrogen, alkyl, alkoxy or halo;

or R⁴, R⁵ of the piperazine-ring are bound to opposite carbon atoms and form a methylene bridge via 1 or 2 methyl groups;

Ar is aryl, and said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro,

wherein the alkyl may be further substituted with 1 to 3 substituents independently selected from the group consisting of halo, amino, alkyl amino, hydroxy, and aryl,

or two of the substituents form together with the carbon atoms they are bound to a 1,3-dioxolane, or a cyclopentane ring, or a cyclohexane ring, and the third remaining substituent may be independently chosen from the substituents described above for alkyl;

R⁶, R⁷, R⁸ are independently chosen from hydrogen, alkyl, alkoxy, alkyl thio, formyl, carboxyl, alkyl carbonyl, alkoxy carbonyl, amino carbonyl, trifluoromethyl, halo, cyano, hydroxy, or nitro.
27a. The in vitro method of the sentences 23a and 26a, wherein said 3,4-dihydroquinazoline is a compound according to Formula (Z1):

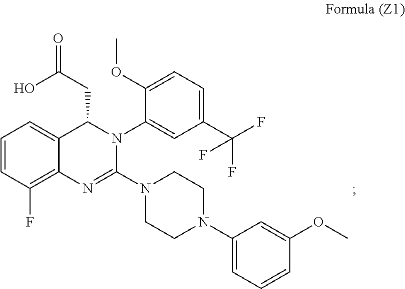 [SMILES: COc1cccc(N2CCN(C3=Nc4c(F)cccc4[C@H](CC(=O)O)N3c3cc(C(F)(F)F)ccc3OC)CC2)c1]

### i.e., Letermovir.

28a. The in vitro method of any of the sentences 23a to 27a, wherein said genetic material provided in step (1) of sentence 23a is viral DNA.
29a. The in vitro method of any of the sentences 23a to 28a, wherein in step (2) of sentence 23a said genetic material is screened for such at least one mutation being located in a ORF UL56 section encoding amino acids at positions 200 to 400 numbered in accordance with the UL56 protein of wild type HCMV (“Merlin”).
30a. The in vitro method of any of the sentences 23a to 28a, wherein in step (2) of sentence 23a said genetic material is screened for such at least one mutation being located in a ORF UL56 section encoding amino acids at positions 230 to 370 numbered in accordance with the UL56 protein of wild type HCMV (“Merlin”).
31a. The in vitro method of any of the sentences 23a to 28a, wherein in step (2) of sentence 23a said genetic material is screened for such at least one mutation being located in a ORF UL56 section comprising nucleotides at positions 598 to 1200 numbered in accordance with the UL56 ORF of HCMV AD169.
32a. The in vitro method of any of the sentences 23a to 28a, wherein in step (2) of sentence 23a said genetic material is screened for such at least one mutation being located in a ORF UL56 section comprising nucleotides at positions 691 to 1107 numbered in accordance with the UL56 ORF of HCMV AD169.
33a. The in vitro method of any of the sentences 23a to 28a, wherein in step (2) of sentence 23a said genetic material is screened for such at least one mutation in the ORF UL56 resulting in an amino acid substitution at a position, numbered in accordance with the UL56 protein of wild type HCMV (Merlin), which is selected from the group consisting of: 231, 232, 236, 241, 325, 369.
34a. The in vitro method of sentence 33a, wherein said position 231 is substituted with a leucine (V231L).
35a. The in vitro method of sentence 33a, wherein said position 232 is substituted with an aspartic acid (N232D).
36a. The in vitro method of sentence 33a, wherein said position 236 is substituted with a methionine (V236M).
37a. The in vitro method of sentence 33a, wherein said position 241 is substituted with a proline (L241P).
38a. The in vitro method of sentence 33a, wherein said position 325 is substituted with a tyrosine (C325Y).
39a. The in vitro method of sentence 33a, wherein said position 369 is substituted with an amino acid selected from the group consisting of: serine (R369S), methionine (R369M), and glycine (R369G).
40a. The in vitro method according to any of the sentences 23a to 28a, wherein step (2) of sentence 23a is realized by means of sequencing the ORF UL56 of the genetic material.
41a. The in vitro method according to any of the sentences 23a to 28a, wherein step (1) is preceded by the following step:
(1′) isolating genetic material of the HCMV from the subject.
42a. The in vitro method according to any of the sentences 23a to 41a, wherein a positive finding under step (3) of sentence 23a indicates a reduced therapeutic susceptibility of a subject infected by HCMV to a 3,4-dihydroquinazoline or a compound of Formula (X) and the administration of said 3,4-dihydroquinazoline or said compound of Formula (X) for use in a method of treatment of HCMV infection is not recommended; and wherein a negative finding under step (3) of sentence 23a indicates no altered therapeutic susceptibility of a subject infected by HCMV to a 3,4-dihydroquinazoline or a compound of Formula (X) and the administration of said 3,4-dihydroquinazoline or said compound of Formula (X) for use in a method of treatment of HCMV infection is recommended.

Exemplary embodiments of the invention are explained in more detail in the following description and are represented in the drawings.

### BRIEF DESCRIPTION OF THE DRAWINGS

FIG. 1 shows the locations of the UL56 amino acid mutations associated with Letermovir and the compound of Formula (X) resistances.

### DESCRIPTION OF PREFERRED EMBODIMENTS

### Materials and Methods

### Synthesis of 3,4-dihydroquinazoline including Letermovir

The syntheses of 3,4-dihydroquinazolines are disclosed in US 2007/0191387 A1. The precise chemical name of Letermovir is (S)-{8-Fluoro-2-[4-(3-methoxyphenyl)-1-piperazinyl]-3-[2-methoxy-5-(trifluoromethyl)phenyl]-3,4-dihydro-4-quinazolinyl}acetic acid. The synthesis of such substance is disclosed in US 2007/0191387 A1, exemplary embodiments 14 and 15, pages 40 and 41, paragraphs [0495] to [0505].

Synthesis of the Compound of Formula (X)

The synthesis of the compound of Formula (X) which precise chemical name is N-{3-[({4-[5-(6-Aminopyridin-2-yl)-1,2,4-oxadiazol-3-yl]phenyl}sulfonyl)amino]-5-fluorophenyl}-1-cyanocyclopropanecarboxamide, is disclosed in US 2009/0176842, exemplary embodiment 1, page 15, paragraphs [0298] to [0302].

Cells, Cell Culture, and Viruses

Normal human dermal fibroblast cells (NHDF; No. CC-2511), human lung fibroblast cells (MRC5; No. CCL-171), and HELF human embryonic lung fibroblast cells (HEL299; No. 87042207) were purchased from Clonetics, the American Type Culture Collection (ATCC), and the European Collection of Cell Cultures (ECACC), respectively, and were cultured. HFF cells were prepared from human foreskin tissue. The HCMV strains AD169 and “Merlin” were purchased from the ATCC (ATCC VR 538, VR-807, and VR-1590), and the AD169-derived recombinant virus RV-HG was reconstituted from the HCMV BAC pHG. For single-step growth curves, 1.5×10⁵ NHDF cells seeded in 12-well dishes were infected in triplicate at a multiplicity of infection (MOI) of 0.1 PFU/cell. Cell supernatants were collected at 24-h intervals for 8 days and stored at −80° C. until the end of the experiment. The virus titer was determined by the immediate-early antigen staining of infected cell nuclei.

Antiviral Assays

HCMV Cytopathic Effect Reduction Assay (CPE-RA)

CPE-RAs were performed basically as described elsewhere (HCMV replication assay). In brief, the addition of 2 μl test compound of 50, 5, 0.5, 0.05, 0.005, and 0.0005 mM DMSO stock solutions to 100 μl cell culture medium in duplicate was followed by serial 2-fold dilutions in 96-well microtiter plates. Each well was supplemented with 150 μl of either a suspension of 1×10⁴ NHDF cells mixed with cell-free HCMV (multiplicity of infection (MOI), 0.03) or a suspension of 1×10⁴ to 3×10⁴ HCMV-infected and uninfected NHDF cells (MOI, 0.001 to 0.002). Non-infected and non-treated cells served as controls on each plate. Final compound concentrations ranged between 250 and 0.00005 μM. Plates were incubated for 6 to 7 days at 37° C. or until the virus control reached 100% CPE. A mixture containing 20% Giemsa stain (Merck) and 5% formalin solution (Merck) was added to the wells for fixation and staining. After extensive washing, plates were dried at 56° C. followed by visual evaluation using an overhead microscope (plaque magnifier; Tecnorama Zürich). Each assay was performed at least in triplicate, and standard deviations were calculated. The assay plate data were used to calculate the EC₅₀ (CPE-RA), i.e., the concentration of drug that inhibits the CPE by 50% compared with an untreated virus-infected control.

HCMV Plaque Assay

HCMV plaque assays were performed as described previously. NHDF cells (1×10⁵ to 2×10⁵) seeded in 24-well tissue culture plates were infected by inoculating 0.1 ml of serial log dilutions of a suspension of infected and uninfected cells. After a 16-h adsorption period, the cell culture supernatant was replaced by 1 ml of a methylcellulose (MC) overlay medium (0.5% MC-Dulbecco modified Eagle medium (DMEM)-10% fetal calf serum (FCS)). Cultures were incubated for 7 to 14 days. Plates were fixed and stained as described above. Subsequently, plates were visually evaluated by counting plaques. Results are expressed as PFU per ml titrated cell suspension.

HCMV Fluorescence Reduction Assay (GFP-RA)

The susceptibilities of recombinant HCMV laboratory strains expressing GFP were determined by a GFP-based fluorescence reduction assay. For standard assays, 1.5×10⁴ NHDF cells/well were cultured in black 96-well plates (Greiner Bio-One, Germany) and infected with either HCMV-GFP (MOI, 0.1) or RV-HG (MOI, 0.2 to 0.5). After virus adsorption, the virus inoculum was replaced with 200 μl fresh medium. Thereafter, 100 μl medium containing the test compounds was added to wells of horizontal row G, followed by serial 3-fold dilutions up to row A. All drug concentrations were tested at least in duplicate. Wells of the horizontal row H served as the virus control. Plates were incubated at 37° C. for 7 days. The medium was replaced by 200 μl PBS, and GFP units (GFPU) were determined by a charge-coupled-device-camera-based fluorescence detector (FluoBox; Bayer Technology Services GmbH, Leverkusen, Germany), which captures 96 images simultaneously. Drug effects were calculated as a percentage of reduction in GFPU in the presence of each drug concentration compared to the GFPU determined in the absence of drug. EC₅₀ and EC₉₀ values (drug concentrations producing 50% and 90% reduction in GFPU) were calculated using nonlinear regression curve fit with a variable slope. GraphPad Prism 3.02 or 4.0 software (GraphPad Software Inc., La Jolla, Calif.) was used for all analyses.

Selection of Resistant Mutants

Drug-resistant HCMV AD169 mutants were selected by the single-step selection method or by serial passage of the virus in the presence of increasing concentration of Letermovir. Briefly, 5×103 AD169-infected NHDF cells/well (MOI, 0.03 PFU/cell) were seeded into the wells of 30 96-well microtiter plates. The infection was allowed to proceed under the exposure of 50 nM Letermovir (˜10×EC₅₀) until a CPE developed in one or more of the compound-treated wells (indicative of resistant virus breakthrough). Non-infected and non-treated cells served as controls on each plate. Mutant virus amplification was accomplished after cultures achieved maximum CPE by the passage of cell-free supernatant virus in the presence of 50 nM Letermovir. The resultant Letermovir-resistant progeny virus mutants were plaque purified three times by limiting dilutions in the presence of Letermovir. The stability of resistance was tested by serially passaging plaque-purified viruses without selective pressure (8 to 10 times). The resistant phenotype of the mutants was confirmed by CPE reduction assays. The sequencing of the open reading frames (ORFs) UL56, UL89, and UL104 revealed the respective UL56 mutations.

En Passant BAC Mutagenesis and Reconstitution of Virus Mutants

The HCMV AD169-derived bacterial artificial chromosome (BAC) pHG served as the backbone for the construction of the mutant viruses. The markerless introduction of point mutations into pHG was done using a two-step recombination protocol according to the en passant method. Briefly, a recombination fragment containing the I-SceI-AphAI cassette from plasmid pEPKan-S was obtained by PCR using primers containing ˜60 bp of homology to the intended integration site in ORF UL56 of BAC pHG and ˜20 bp specific for pEPKan-S. This fragment was electroporated into Escherichia coli GS1783 that harbored the pHG BAC. The first Red recombination resulted in a selectable BAC with an ISceI restriction site and a kanamycin cassette flanked by a duplication of the UL56 target sequence. After successful kanamycin selection, all non-HCMV sequences were removed by an intrabacterial ISceI digest and a subsequent second Red recombination, resulting in the scarless repair of the mutated UL56 gene in the background of BAC pHG. The integrity of the generated BACs was confirmed by restriction enzyme digestion and sequencing. To reconstitute virus mutants, the recombinant BACs were transfected into permissive MRC5 cells. Reconstituted mutant viruses were termed as indicated in tables 6 and 7. At least two independent virus clones per mutation were generated and evaluated.

Amplification of the UL56 Letermovir Mutation Hot Spot Via Nested PCR Using Two Airs of Primers

Primer Sequences

TABLE 1
Amplification PCR primers

| Int.- | | | SEQ | Tm- | Annealing | Amplificate | |
|---|---|---|---|---|---|---|---|
| No: | Name | Sequence | ID NO: | Values | Temp: | (bp) | Elongation |
| 5-82 | 5′UL56- | ACGCGGCGGCTATAGTGTAT | 5 | 54.0° C. | 50° C. | 1961 | 2.5 min |
| | Ext2 | | | | | | |
| 5-83 | 3′UL56- | GCATCCACATCTCCTTCTGC | 6 | 51.9° C. | | | |
| | Ext2 | | | | | | |
| 6-1 | 5′UL56- | AGCTGACCATCATCCCGAAT | 7 | 53.2° C. | 51° C. | 1328 | 1.5 min |
| | Int2 | | | | | | |
| 6-2 | 3′UL56- | CCACATTGTGAGAGAGGGGATA | 8 | 53.0° C. | | | |
| | Int2 | | | | | | |

The external primer pair 5-82 and 5-83 was used to amplify UL56 nt132-nt2092 from HCMV infected cell culture or patient specimen. Following the first amplification the second internal PCR was performed using primer pairs 6-1 and 6-2 in order to obtain UL56 nt691-nt1107. 1-5 μl of the product of the first PCR was added as a template to the second PCR reaction.

The nested PCR was performed under the following conditions:

| First PCR using primer | | | Second PCR using | | |
|---|---|---|---|---|---|
| pair 5-82/5-83: | | | primer pair 6-1/6-2: | | |
| 95° C. | 5 min | | 95° C. | 5 min | |
| 95° C. 50° C. | 20 s 30 s | 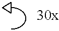 | 95° C. 51° | 20 s 30 s | 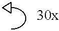 |
| 72° C. | 2:30 min | | 72° C. | 1:30 min | |
| 72° C. | 3:00 min | | 72° C. | 3:00 min | |
| 4° C. | HOLD | | 4° C. | HOLD | |

Polymerase: Platinum Pfx polymerase; INVITROGEN; catalog number: 11708-021

Primer: Eurogentec; concentration of all stock solutions 10 μM

dNTP's: PCR Nucleotide Mix (10×200 μl); Roche; catalog number: 11 814 362 001

### Standard PCR Sample (all in μl):

TABLE 2
Standard PCR Sample

| | 1x |
|---|---|
| Template | 1-5 |
| 5′ primer (10 μM) | 1 |
| 3′ primer (10 μM) | 1 |
| dNTPs (10 mM) | 1 |
| Platinum Pfx Poly. (2.5 U/μl) | 1 |
| Platinum Pfx Buffer (10x) | 5 |
| MgSO₄ (50 mM) | 1 |
| Enhancer | 1 |
| Ampuwa Water | ad 50 |

For the sequencing of full-length UL56 the following primers are required:

TABLE 3
Sequencing PCR primers

| | | | SEQ | |
|---|---|---|---|---|
| Int.-No: | Name: | Sequence | ID NO: | Sense/Antisense |
| 1-27 | 3UL56-1291 | GTCCACGCAGGTGAATAGCC | 9 | A |
| 1-61 | 5UL56 genomic | AAACCGCAGCACCCAGACAG | 10 | S |
| 4-60 | 5′UL56 252 | TACGGCTTTGCTGGATCGGG | 11 | S |
| 1-28 | 5UL56-539 | CGCGTCAGGAAGTGTACGTC | 12 | S |
| 4-62 | 5′UL56 881 | TGTCGGATTTTACCTACTGGTC | 13 | S |
| 4-63 | 5′UL56 1181 | GCAACGAGATGTACACCAAAATC | 14 | S |
| 1-30 | 3UL56-1342 | TGCTGTTGCTGGCCGTCATC | 15 | A |
| 2-41 | 5′UL56 1582 | GAGGTGGGCTATGGCAAGGT | 16 | S |
| 4-65 | 5′UL56 1785 | TGTGGTCAATAACCTGATCCA | 17 | S |
| 2-42 | 5′UL56 1883 | ACTGCACCGATCGTTATCCC | 18 | S |
| 4-66 | 5′UL562081 | AGATGTGGATGCACGTGCGG | 19 | S |
| 2-43 | 3′UL56 2254 | GGAGATATAGATCTTTGGACTT | 20 | A |
| 4-67 | 5′UL562369 | TCTCGGGTGTCTTCAGGGAG | 21 | S |
| 1-62 | 3UL56-genomic | AGCGGCCCGCGAGTTATTTG | 22 | A |

The primers held in italic letters are required for the UL hot spot sequencing of nt691-nt 1107.

Results

Identification of Mutations Putatively Conferring Resistance to Letermovir

The inventors screened the HCMV strains resistant to Letermovir, respectively, for mutations in the ORFs UL56, UL89, UL104, and UL51. As depicted in Table 4, it turned out that the resistant viruses harbored only distinct point mutations in ORF UL56, implicating this protein as being involved in the Letermovir mechanism of action.

TABLE 4
Pheno- and genotypic characterization of letermovir resistant HCMV mutants obtained in vitro

| HCMV | EC₅₀ [μM] [symbol] | | | mutation (aa) [symbol] | | | | marker |
|---|---|---|---|---|---|---|---|---|
| strain | letermovir | GCV | RI [symbol] | UL56 [symbol] | UL89 [symbol] | UL104 [symbol] | UL51 [symbol] | transfer |

| AD169 | 0.0056 ± 0.0016 | 3.4 ± 1.5 | — | — | — | — | — | − |
|---|---|---|---|---|---|---|---|---|
| selected mutants [symbol] | | | | | | | | |
| rAIC246-1 | 1.24 ± 0.36 | 1.2 ± 0.2 | 221 | L241P | — | — | — | + |
| rAIC246-2 | 0.37 ± 0.07 | 4.0 ± 0.9 | 66 | R369S | — | — | — | + |
| rAIC246-3 | 27 ± 3.27 | 2.4 ± 2.5 | 4801 | C325Y | — | — | — | + |
| rAIC246-4 | 0.13 ± 0.01 | 4.2 ± 1.3 | 23 | V231L | — | — | — | − |
| rAIC246-5 | 0.11 ± 0.01 | 5.0 ± 0.4 | 19 | R369M | — | — | — | + |
| rAIC246-6 | compare rAIC246-5 | | | R369M | — | — | — | + |
| rAIC246-7 | compare rAIC246-1 | | | L241P | — | — | — | + |
| rAIC246-8 | compare rAIC246-3 | | | C325Y | — | — | — | + |
| rAIC246-9 | 0.06 ± 0.04 | 1.7 ± 0.2 | 11 | R369G | — | — | — | + |
| rAIC246-10 | 0.07 ± 0.02 | 1.7 ± 0.3 | 13 | V236M | — | — | — | − |

| | | | | |
|---|---|---|---|---|
| rAIC246-11 | n.d. | n.d. | N232D | n.d. |

^{a}EC₅₀ values determined by a CPE reduction assay. Data are means of at least three independent experiments and are expressed ±standard deviation.
^{b}Resistance index (RI) = letermovir-EC₅₀ mutant virus/letermovir-EC₅₀ wild-type virus.
^{c}amino acid exchange identified by HCMV genotyping
^{d}HCMV genes involved in cleavage/packaging of viral progeny DNA
^{e}HCMV strain AD169 virus mutants obtained in vitro under selective pressure with letermovir [symbol]
indicates data missing or illegible when filed

TABLE 5
Pheno- and genotypic characterization of letermovir resistant HCMV mutants obtained in vitro (update)

| HCMV | EC₅₀ [μM] [symbol] | | | mutation (aa) [symbol] | | | | marker |
|---|---|---|---|---|---|---|---|---|
| strain | letermovir | GCV | RI [symbol] | UL56 [symbol] | UL89 [symbol] | UL104 [symbol] | UL51 [symbol] | transfer |
| AD169 | 0.0056 ± 0.0016 | 3.4 ± 1.5 | — | — | — | — | — | − |
| selected mutants [symbol] | | | | | | | | |
| rAIC246-1 | 1.24 ± 0.38 | 1.2 ± 0.2 | 221 | L241P | — | — | — | + |
| rAIC246-2 | 0.37 ± 0.07 | 4.0 ± 0.9 | 66 | R369S | — | — | — | + |
| rAIC246-3 | 27 ± 3.27 | 2.4 ± 2.5 | 4801 | C325Y | — | — | — | + |
| rAIC246-4 | 0.13 ± 0.01 | 4.2 ± 1.3 | 23 | V231L | — | — | — | + |
| rAIC246-5 | 0.11 ± 0.01 | 5.0 ± 0.4 | 19 | R369M | — | — | — | + |
| rAIC246-6 | 0.08 ± 0.02 | 2.9 ± 0.9 | 14 | R369M | — | — | — | + |
| rAIC246-7 | 0.92 ± 0.12 | 2.2 ± 0.6 | 164 | L241P | — | — | — | + |
| rAIC246-8 | 25 ± 5.53 | 2.2 ± 1.2 | 4428 | C325Y | — | — | — | + |
| rAIC246-9 | 0.06 ± 0.04 | 1.7 ± 0.2 | 11 | R369G | — | — | — | + |
| rAIC246-10 | 0.07 ± 0.02 | 1.7 ± 0.3 | 13 | V236M | — | — | — | + |

| | | | | |
|---|---|---|---|---|
| rAIC246-11 | n.d. | n.d. | N232D | n.d. |

^{a}EC₅₀ values determined by a CPE reduction assay. Data are means of at least three independent experiments and are expressed ±standard deviation.
^{b}Resistance index (RI) = letermovir-EC₅₀ mutant virus/letermovir-EC₅₀ wild-type virus.
^{c}amino acid exchange identified by HCMV genotyping
^{d}HCMV genes involved in cleavage/packaging of viral progeny DNA
^{e}HCMV strain AD169 virus mutants obtained in vitro under selective pressure with letermovir [symbol]
indicates data missing or illegible when filed

As depicted in Table 5, the inventors also obtained phenotyping data of virus variants rAIC246-6, rAIC246-7 and rAIC246-8. Additionally, the marker transfer for all selected mutants was positive indicating that all putative resistance mutations were confirmed by marker transfer experiments (see hereto Tables 6, 7 and 8 below).

Recombinant Phenotyping by Marker Transfer

To obtain direct evidence that the identified UL56 mutations were necessary and sufficient to cause resistance to Letermovir, the marker transfer of the respective mutations to HCMV strain AD169 was performed. Applying markerless BAC mutagenesis, the inventors independently introduced the discovered UL56 nucleotide mutations into the GFP-expressing, AD169-derived BAC pHG. The structural integrity of the resulting BACs pHG-UL56-L241P and pHG-UL56-R369S was examined by restriction enzyme cleavage and sequencing. Infectious virus was reconstituted by transfecting BAC DNA into permissive MRC5 cells. The inventors then characterized the growth properties of the obtained recombinant viruses in one-step growth curves. The parental virus RV-HG and all tested virus mutants exhibited similar growth kinetics in these experiments. Hence, the putative resistance mutations had little or no effect on the growth properties of the recombinants in vitro.

Likewise the structural integrity of all resulting BACs were examined by restriction enzyme cleavage and sequencing. Transfecting the BAC DNA into permissive MRC5 cells reconstituted infectious virus. The inventors then characterized the growth properties of the obtained recombinant viruses in one-step growth curves. The parental virus RV-HG and all tested virus mutants exhibited similar growth kinetics in these experiments. Hence, the putative resistance mutations had little or no effect on the growth properties of the recombinants in vitro.

To precisely elucidate the role of the UL56 mutations in drug resistance, the inventors next determined the drug susceptibility profile for the newly constructed virus mutants by fluorescence reduction assays. The data summarized in Tables 6 and 7 show that the RV-HG-UL56 mutants were resistant to Letermovir. E.g., mutation L241P conferred a ˜160-fold increase and mutation R369S a ˜38-fold increase in the respective EC₅₀s. The sensitivity of the mutants to approved control compounds that act by a different mechanism (GCV, CDV, and FOS) did not differ from the sensitivity of the parental wild-type virus RV-HG (Tables 6 and 7). The finding that mutations within one terminase subunit confer resistance to Letermovir prompted the inventors to examine whether the Letermovir-resistant viruses demonstrate cross-resistance against sulfonamides (e.g., BAY 38-4766) or benzimidazoles (e.g., BDCRB), the only two chemical classes of HCMV inhibitors that were reported to target the viral terminase. Interestingly, the inventors noticed that both compounds were comparably active against the parental wild-type HCMV (RV-HG) as well as against all Letermovir-resistant strains (Tables 6 and 7) with the exception of a single strain that carried the mutation UL56 R369M. Taken together, these findings (i) confirm that a single conservative amino acid substitution is necessary and sufficient to produce Letermovir resistance in vitro, (ii) suggest that Letermovir inhibits HCMV replication through a specific antiviral mechanism that involves the viral gene product UL56, and (iii) imply that Letermovir exerts its effects at the molecular level via a mechanism that is distinct from that of other compound classes known to target the HCMV terminase.

TABLE 6
Marker transfer - Susceptibility of reconstituted wt-virus and generated UL56 mutant viruses to Letermovir,
approved and developmental HCMV drugs including drugs targeting the viral terminase - Part I

| | EC₅₀ [symbol] [μM] | | | | | | | | |
|---|---|---|---|---|---|---|---|---|---|
| | RV-HG | RV-HG | | RV-HG | | RV-HG | | RV-HG | |
| Drug | (wt-HCMV) | UL56-C325Y | RI [symbol] | UL56-C325Yrev | RI | UL56-L241P | RI | UL56-V236M | RI |
| Letermovir | 0.0030 ± 0.0012 | 26.4 ± 8.5 | 8910 | 0.0039 ± 0.0009 | — | 0.68 ± 0.24 | 221 | 0.07 ± 0.02 | 24 |
| Formula (X) | 0.0141 ± 0.0079 | 0.0367 ± 0.0039 | — | 0.0317 ± 0.0024 | — | 0.0183 ± 0.0100 | — | 0.0086 ± 0.0013 | — |
| Ganciolovir [symbol] | 2.21 ± 0.99 | 2.11 ± 0.71 | — | 2.83 ± 0.81 | — | 2.11 ± 0.77 | — | 1.56 ± 0.33 | — |
| Cidofovir [symbol] | 0.25 ± 0.17 | 0.35 ± 0.05 | — | 0.32 ± 0.13 | — | 0.21 ± 0.09 | — | 0.26 ± 0.18 | — |
| Foscarnet [symbol] | 111 ± 55 | 126 ± 20 | — | 178 ± 12 | — | 115 ± 71 | — | 76 ± 35 | — |
| Aciciovir [symbol] | 74 ± 40 | 49 ± 16 | — | 55 ± 18 | — | 53 ± 12 | — | 80 ± 21 | — |
| BAY384766 [symbol] | 0.41 ± 0.15 | 1.07 ± 0.14 | — | 0.58 ± 0.06 | — | 0.34 ± 0.14 | — | 0.33 ± 0.08 | — |
| BDCRB [symbol] | 0.33 ± 0.12 | 0.30 ± 0.04 | — | 0.29 ± 0.04 | — | 0.27 ± 0.04 | — | 0.17 ± 0.03 | — |
| Maribavir [symbol] | 0.18 ± 0.11 | 0.29 ± 0.14 | — | 0.18 ± 0.07 | — | 0.14 ± 0.07 | — | n.d. [symbol] | — |

^{a}EC₅₀ values determined by a fluorescence reduction assay. Data are means of at least three independent experiments and are expressed ±standard deviation.
^{b}Resistance index (RI) = EC₅₀ indicated RV-HG mutant/EC₅₀ RV-HG wt
^{c}approved polymerase inhibitor
^{d}discontinued terminase inhibitor
^{e}developmental UL97 inhibitor
^{f}not determined; Formula (X) indicates a compound of Formula (X) [symbol]
indicates data missing or illegible when filed

TABLE 7
Marker transfer - Susceptibility of reconstituted wt-virus and generated UL56 mutant viruses to Letermovir,
approved and developmental HCMV drugs including drugs targeting the viral terminase - Part II

| | EC₅₀ [symbol] [μM] | | | | | | |
|---|---|---|---|---|---|---|---|
| | RV-HG | RV-HG | | RV-HG | | RV-HG | |
| Drug | (wt-HCMV) | UL56-R369S | RI [symbol] | UL56-R369M | RI | UL56-L241P/R369S | RI |
| Letermovir | 0.0030 ± 0.0012 | 0.14 ± 0.07 | 48 | 0.040 ± 0.015 | 13 | 5.26 ± 0.00 | 1776 |
| Formula (X) | 0.0141 ± 0.0079 | 0.0295 ± 0.0137 | 2 | 0.096 ± 0.018 | 7 | 0.0416 ± 0.0180 | 3 |
| Ganciolovir [symbol] | 2.21 ± 0.99 | 1.96 ± 1.09 | — | 1.26 ± 0.64 | — | 2.86 ± 1.16 | — |
| Cidofovir [symbol] | 0.25 ± 0.17 | 0.16 ± 0.05 | — | 0.18 ± 0.11 | — | 0.20 ± 0.11 | — |
| Foscarnet [symbol] | 111 ± 55 | 61 ± 21 | — | 76 ± 25 | — | 62 ± 30 | — |
| Aciciovir [symbol] | 74 ± 40 | 45 ± 10 | — | 88 ± 29 | — | 81 ± 33 | — |
| BAY384766 [symbol] | 0.41 ± 0.16 | 0.68 ± 0.13 | — | 1.75 ± 0.16 | 4 | 0.84 ± 0.31 | 2 |
| BDCRB [symbol] | 0.33 ± 0.12 | 0.19 ± 0.02 | — | 0.11 ± 0.05 | — | 0.30 ± 0.14 | — |
| Maribavir [symbol] | 0.18 ± 0.11 | 0.08 ± 0.03 | — | n.d. | — | n.d. | — |

^{a}EC₅₀ values determined by a fluorescence reduction assay. Data are means of at least three independent experiments and are expressed ±standard deviation.
^{b}Resistance index (RI) = EC₅₀ indicated RV-HG mutant/EC₅₀ RV-HG wt
^{c}approved polymerase inhibitor
^{d}discontinued terminase inhibitor
^{e}developmental UL97 inhibitor
^{f}not determined; Formula (X) indicates a compound of Formula (X) [symbol]
indicates data missing or illegible when filed

TABLE 8
Marker transfer - Susceptibility of reconstituted wt-
virus and generated UL56 mutant viruses to Letermovir,
approved and developmental HCMV drugs including drugs
targeting the viral terminase - Part III

| | EC₅₀ [symbol] [μM] | | |
|---|---|---|---|
| | RV-HG | RV-HG | |
| Drug | (wt-HCMV) | UL56-V231L | RI [symbol] |
| Letermovir | 0.0030 ± 0.0012 | 0.02 ± 0.0047 | 5 |
| Formula (X) | 0.0141 ± 0.0079 | 0.0071 ± 0.0012 | — |
| Ganciclovir [symbol] | 2.21 ± 0.99 | 0.72 ± 0.24 | — |
| Cidofovir [symbol] | 0.25 ± 0.17 | 0.09 ± 0.02 | — |
| Foscamet [symbol] | 111 ± 55 | 67 ± 18 | — |
| Aciclovir [symbol] | 74 ± 40 | 29 ± 9 | — |
| BAY384766 [symbol] | 0.41 ± 0.15 | 0.36 ± 0.08 | — |
| BDCRB [symbol] | 0.33 ± 0.12 | 0.21 ± 0.04 | — |
| Maribavir [symbol] | 0.18 ± 0.11 | n.d. [symbol] | — |

^{a}EC [symbol] values determined by a fluorescence reduction assay. Data are means of at least three independent experiments and are expressed ± standard deviation.
^{b}Resistance index (RI) = EC [symbol] indicated RV-HG mutant/EC [symbol] RV-HG wt
^{c}approved polymerase inhibitor
^{d}discontinued terminase inhibitor
^{e}developmental UL97 inhibitor
^{f}not determined; Formula (X) indicates a compound of Formula (X) [symbol]
indicates data missing or illegible when filed

Table 8 summarizes the results of UL56 V231L marker transfer experiments that were not included in Tables 6 and 7.

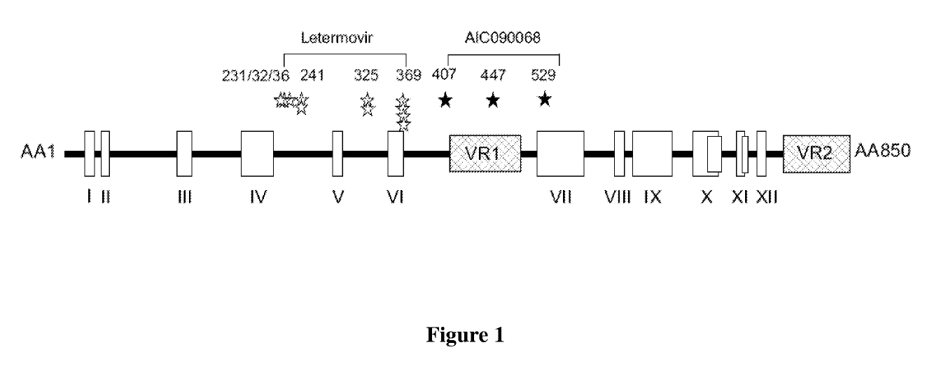

FIG. 1 depicts a schematic representation of the HCMV UL56 domain organization according to Champier, et al., “Putative functional domains of human cytomegalovirus pUL56 involved in dimerization and benzimidazole D-ribonucleoside activity.” Antivir. Ther. 2008, 13, pages 643-654. Conserved regions are indicated as white boxes (I-XII); variable regions (VR1 and VR2) as hatched boxes. The Letermovir mutation hot-spot (amino acids 230-370) and positions of aa associated with in vitro resistance against Letermovir are indicated (white stars, aa 231, 232, 236, 241, 325 and 369). In addition, UL56 mutations are indicated that putatively confer drug resistance to the drug candidate of the formula (X) (black stars; aa 369, 407, 447, 529, and 662).

Sequences

SEQ ID No. 1: amino acid sequence of wild type (“Merlin”) UL56 protein.

SEQ ID No. 2: amino acid sequence of the laboratory HCMV stem AD169 UL56 protein

SEQ ID No. 3: nucleotide sequence encoding wild type (“Merlin”) UL56 protein

SEQ ID No. 4: nucleotide sequence encoding laboratory HCMV stem AD169 UL56 protein

SEQ ID Nos. 5-8: UL56 amplification PCR primers

SEQ ID Nos. 9-22: UL56 sequencing PCR primers

1. A method for the identification of an altered therapeutic susceptibility of a subject infected by HCMV to a treatment with a 3,4 dihydroquinazoline or a compound of Formula (X) 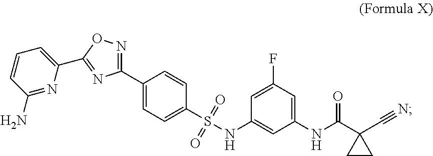 [SMILES: N#CC1(C(=O)Nc2cc(F)cc(NS(=O)(=O)c3ccc(-c4noc(-c5cccc(N)n5)n4)cc3)c2)CC1]
i.e., N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide,
comprising the following steps:
(1) providing genetic material of the HCMV isolated from said subject,
(2) screening said genetic material for at least one mutation in the ORF UL56, and
(3) correlating a positive finding in step (2) with the presence of a reduced therapeutic susceptibility.

2. The method of claim 1, wherein the altered therapeutic susceptibility is a reduced therapeutic susceptibility.

3. The method of claim 1, wherein said 3,4 dihydroquinazoline is Letermovir.

4. The method of claim 1, wherein said genetic material provided in step (1) is viral DNA.

5. The method of claim 1, wherein in step (2) said genetic material is screened for such at least one mutation being located in a ORF UL56 section encoding amino acids at positions 200 to 400 numbered in accordance with the UL56 protein of wild type HCMV (“Merlin”).

6. The method of claim 1, wherein in step (2) said genetic material is screened for such at least one mutation being located in a ORF UL56 section encoding amino acids at positions 230 to 370 numbered in accordance with the UL56 protein of wild type HCMV (“Merlin”).

7. The method of claim 1, wherein in step (2) said genetic material is screened for such at least one mutation being located in a ORF UL56 section comprising nucleotides at positions 598 to 1200 numbered in accordance with the UL56 ORF of HCMV AD169.

8. The method of claim 1, wherein in step (2) said genetic material is screened for such at least one mutation being located in a ORF UL56 section comprising nucleotides at positions 691 to 1107 numbered in accordance with the UL56 ORF of HCMV AD169.

9. The method of claim 1, wherein in step (2) said genetic material is screened for such at least one mutation in the ORF UL56 resulting in an amino acid substitution at a position, numbered in accordance with the UL56 protein of wild type HCMV (Merlin), which is selected from the group consisting of: 231, 232, 236, 241, 325, 369.

10. The method of claim 9, wherein said position 231 is substituted with a leucine (V231L).

11. The method of claim 9, wherein said position 232 is substituted with an aspartic acid (N232D).

12. The method of claim 9, wherein said position 236 is substituted with a methionine (V236M).

13. The method of claim 9, wherein said position 241 is substituted with a proline (L241P).

14. The method of claim 9, wherein said position 325 is substituted with a tyrosine (C325Y).

15. The method of claim 9, wherein said position 369 is substituted with an amino acid selected from the group consisting of: serine (R369S), methionine (R369M), and glycine (R369G).

16. The method of claim 1, wherein step (2) is realized by means of sequencing the ORF UL56 of the genetic material.

17. The method of claim 1, wherein step (1) is preceded by the following step:
1′. isolating genetic material of the HCMV from the subject.

18. The method of claim 1, wherein said 3,4 dihydroquinazoline is a compound having a backbone according to Formula (Y): 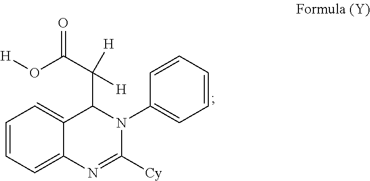
wherein
Cy is selected from a carbocyclic or heterocyclic ring system;
wherein a carbocyclic ring system denotes a saturated hydrocarbon ring group having 5 to 6 ring carbon atoms; a 5- to 6-membered carbocyclic aromatic ring; bicyclic ring systems wherein at least one ring is carbocyclic and aromatic, tricyclic ring systems wherein at least one ring is carbocyclic and aromatic;
wherein a heterocyclic ring system denotes a 5- to 6-membered aliphatic ring as well as a 5- to 6-membered aromatic ring containing one or more heteroatoms chosen from N, O, and S, with the remaining ring atoms being carbon; and
bicyclic heterocycloalkyl rings containing one or more heteroatoms chosen from N, O, and S, with the remaining ring atoms being carbon and wherein at least one heteroatom is present in an aromatic ring;
and wherein all ring systems, except for the central ring having two nitrogen atoms according to Formula (Y), may be further substituted with one or more heteroatoms chosen from N, O, and S.

19. The method of claim 1, wherein said 3,4 dihydroquinazoline is a compound according to any of the Formulae (Y1; Y2; Y3; Y4; Y5): 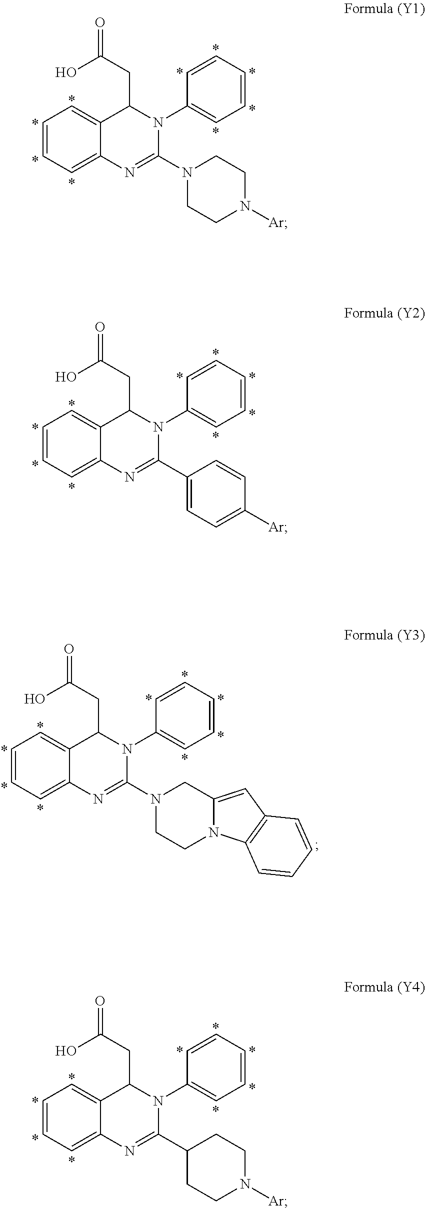 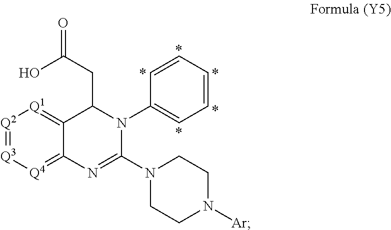
wherein
in any of the Formulae (Y1; Y2; Y3; Y4; Y5) asterisk (*) illustrates potential positions for further substituents; and Ar indicates a further substituent aryl, wherein said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;
and wherein
in Formula (Y5) one or two of Q¹, Q², Q³ and Q⁴ represent N and the others simultaneously represent CH.

20. The method of claim 1, wherein said 3,4 dihydroquinazoline is a compound according to Formula (Z) or a pharmaceutically acceptable salt or solvate thereof or the salt of said solvate thereof: 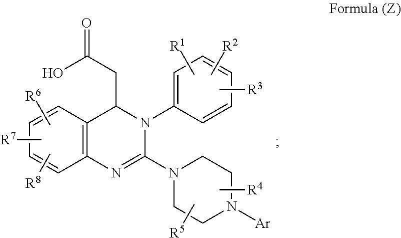
wherein
R¹ is selected from hydrogen, amino, alkyl, alkoxy, alkyl amino, alkyl thio, cyano, halo, nitro, trifluoromethyl, or trifluoromethoxy;
R² is selected from hydrogen, alkyl, alkoxy, alkyl thio, cyano, halo, nitro, or trifluoromethyl;
R³ is selected from amino, alkyl, alkoxy, alkyl amino, alkyl thio, cyano, halo, nitro, trifluoromethyl, alkylsulfonyl, or alkylaminosulfonyl;
or one of R¹, R², R³ is selected from hydrogen, alkyl, alkoxy, cyano, halo, nitro, or trifluoromethyl, and the both remaining substituents form a 1,3 dioxolane, or a cyclopentane ring, or a cyclohexane ring via its carbon atoms that are bound to the ring system;
R⁴, R⁵ are independently chosen from hydrogen, alkyl, alkoxy or halo;
or R⁴, R⁵ of the piperazine ring according to Formula (Z) are bound to opposite carbon atoms and form a methylene bridge via 1 or 2 methyl groups;
Ar is aryl, and said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;
wherein the alkyl may be further substituted with 1 to 3 substituents independently selected from the group consisting of halo, amino, alkyl amino, hydroxy, and aryl,
or two of the substituents form together with the carbon atoms they are bound to a 1,3-dioxolane, or a cyclopentane ring, or a cyclohexane ring, and the third remaining substituent may be independently chosen from the substituents described above for alkyl;
R⁶, R⁷, R⁸ are independently chosen from hydrogen, alkyl, alkoxy, alkyl thio, formyl, carboxyl, alkyl carbonyl, alkoxy carbonyl, amino carbonyl, trifluoromethyl, halo, cyano, hydroxy, or nitro.

21. The method of claim 1, wherein said 3,4 dihydroquinazoline is a compound according to Formula (Z1) or a pharmaceutically acceptable salt or solvate thereof or the salt of said solvate thereof: 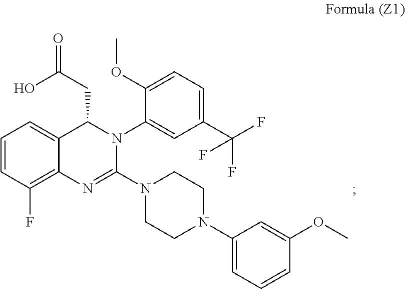 [SMILES: COc1cccc(N2CCN(C3=Nc4c(F)cccc4[C@H](CC(=O)O)N3c3cc(C(F)(F)F)ccc3OC)CC2)c1]
i.e., Letermovir.

22. The method of claim 1, wherein said method is an in vitro method.

23. An in vitro method for use in the identification of an altered therapeutic susceptibility of a subject infected by HCMV to a 3,4 dihydroquinazoline or the compound of Formula (X) 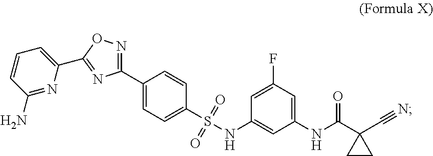 [SMILES: N#CC1(C(=O)Nc2cc(F)cc(NS(=O)(=O)c3ccc(-c4noc(-c5cccc(N)n5)n4)cc3)c2)CC1]
i.e. N-{3-[({4-[5-(6-Aminopyridin-2-yl)-1,2,4-oxadiazol-3-yl]phenyl}sulfonyl)amino]-5-fluorophenyl}-1-cyanocyclopropanecarboxamide,
comprising the following steps:
(1) providing genetic material of the HCMV from a subject infected with HCMV,
(2) screening said genetic material for at least one mutation in the ORF UL56, and
(3) correlating a positive finding in step (2) with a reduced therapeutic susceptibility.

24. The in vitro method of claim 23, wherein said 3,4 dihydroquinazoline is a compound having a backbone according to Formula (Y): 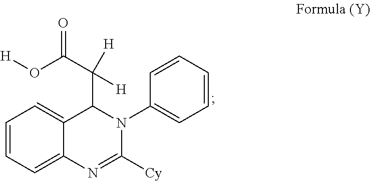
wherein
Cy is selected from a carbocyclic or heterocyclic ring system;
wherein a carbocyclic ring system denotes a saturated hydrocarbon ring group having 5 to 6 ring carbon atoms; a 5- to 6-membered carbocyclic aromatic ring; bicyclic ring systems wherein at least one ring is carbocyclic and aromatic, tricyclic ring systems wherein at least one ring is carbocyclic and aromatic;
wherein a heterocyclic ring system denotes a 5- to 6-membered aliphatic ring as well as a 5- to 6-membered aromatic ring containing one or more heteroatoms chosen from N, O, and S, with the remaining ring atoms being carbon; and
bicyclic heterocycloalkyl rings containing one or more heteroatoms chosen from N, O, and S, with the remaining ring atoms being carbon and wherein at least one heteroatom is present in an aromatic ring;
and wherein all ring systems, except for the central ring having two nitrogen atoms according to Formula (Y), may be further substituted with one or more heteroatoms chosen from N, O, and S.

25. The in vitro method of claim 23, wherein said 3,4 dihydroquinazoline is a compound according to any of the Formulae (Y1; Y2; Y3; Y4; Y5): 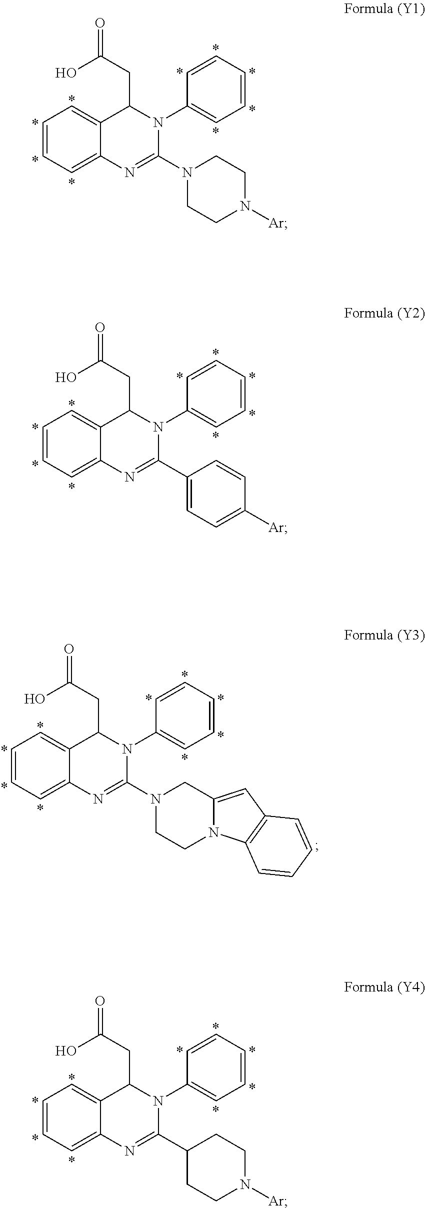 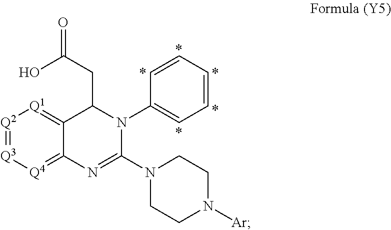
wherein
in any of the Formulae (Y1; Y2; Y3; Y4; Y5) asterisk (*) illustrates potential positions for further substituents; and Ar indicates a further substituent aryl, wherein said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro;
and wherein
in Formula (Y5) one or two of Q¹, Q², Q³ and Q⁴ represent N and the others simultaneously represent CH.

26. The in vitro method of the claim 23, wherein said 3,4 dihydroquinazoline is a compound according to Formula (Z): 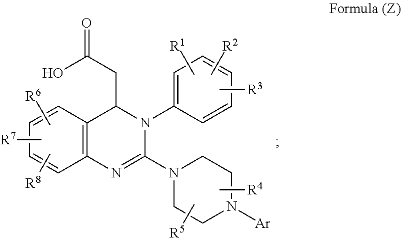
wherein
R¹ is selected from hydrogen, amino, alkyl, alkoxy, alkyl amino, alkyl thio, cyano, halo, nitro, trifluoromethyl, or trifluoromethoxy;
R² is selected from hydrogen, alkyl, alkoxy, alkyl thio, cyano, halo, nitro, or trifluoromethyl;
R³ is selected from amino, alkyl, alkoxy, alkyl amino, alkyl thio, cyano, halo, nitro, trifluoromethyl, alkylsulfonyl, or alkylaminosulfonyl;
or one of R¹, R², R³ is selected from hydrogen, alkyl, alkoxy, cyano, halo, nitro, or trifluoromethyl and the both remaining substituents form a 1,3 dioxolane, or a cyclopentane ring, or a cyclohexane ring via its carbon atoms that are bound to the ring system;
R⁴, R⁵ are independently chosen from hydrogen, alkyl, alkoxy or halo;
or R⁴, R⁵ of the piperazine-ring are bound to opposite carbon atoms and form a methylene bridge via 1 or 2 methyl groups;
Ar is aryl, and said aryl may be further substituted with 1 to 3 substituents independently selected from the group consisting of alkyl, alkoxy, formyl, carboxyl, alkyl carbonyl, alkoxy carbonyl, trifluoromethyl, halo, cyano, hydroxy, amino, alkyl amino, amino carbonyl, and nitro,
wherein the alkyl may be further substituted with 1 to 3 substituents independently selected from the group consisting of halo, amino, alkyl amino, hydroxy, and aryl,
or two of the substituents form together with the carbon atoms they are bound to a 1,3-dioxolane, or a cyclopentane ring, or a cyclohexane ring, and the third remaining substituent may be independently chosen from the substituents described above for alkyl;
R⁶, R⁷, R⁸ are independently chosen from hydrogen, alkyl, alkoxy, alkyl thio, formyl, carboxyl, alkyl carbonyl, alkoxy carbonyl, amino carbonyl, trifluoromethyl, halo, cyano, hydroxy, or nitro.

27. The in vitro method of claim 23, wherein said 3,4 dihydroquinazoline is a compound according to Formula (Z1): 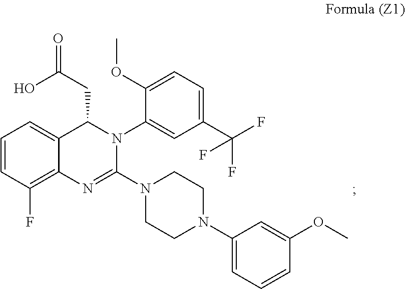 [SMILES: COc1cccc(N2CCN(C3=Nc4c(F)cccc4[C@H](CC(=O)O)N3c3cc(C(F)(F)F)ccc3OC)CC2)c1]
i.e., Letermovir.

28. The in vitro method of claim 23, wherein said genetic material provided in step (1) of claim 23 is viral DNA.

29. The in vitro method of claim 23, wherein in step (2) of claim 23 said genetic material is screened for such at least one mutation being located in a ORF UL56 section encoding amino acids at positions 200 to 400 numbered in accordance with the UL56 protein of wild type HCMV (“Merlin”).

30. The in vitro method of claim 23, wherein in step (2) of claim 23 said genetic material is screened for such at least one mutation being located in a ORF UL56 section encoding amino acids at positions 230 to 370 numbered in accordance with the UL56 protein of wild type HCMV (“Merlin”).

31. The in vitro method of claim 23, wherein in step (2) of claim 23 said genetic material is screened for such at least one mutation being located in a ORF UL56 section comprising nucleotides at positions 598 to 1200 numbered in accordance with the UL56 ORF of HCMV AD169.

32. The in vitro method of claim 23, wherein in step (2) of claim 23 said genetic material is screened for such at least one mutation being located in a ORF UL56 section comprising nucleotides at positions 691 to 1107 numbered in accordance with the UL56 ORF of HCMV AD169.

33. The in vitro method of claim 23, wherein in step (2) of claim 23 said genetic material is screened for such at least one mutation in the ORF UL56 resulting in an amino acid substitution at a position, numbered in accordance with the UL56 protein of wild type HCMV (Merlin), which is selected from the group consisting of: 231, 232, 236, 241, 325, 369.

34. The in vitro method of claim 33, wherein said position 231 is substituted with a leucine (V231L).

35. The in vitro method of claim 33, wherein said position 232 is substituted with an aspartic acid (N232D).

36. The in vitro method of claim 33, wherein said position 236 is substituted with a methionine (V236M).

37. The in vitro method of claim 33, wherein said position 241 is substituted with a proline (L241P).

38. The in vitro method of claim 33, wherein said position 325 is substituted with a tyrosine (C325Y).

39. The in vitro method of claim 33, wherein said position 369 is substituted with an amino acid selected from the group consisting of: serine (R369S), methionine (R369M), and glycine (R369G).

40. The in vitro method of claim 23, wherein step (2) of claim 23 is realized by means of sequencing the ORF UL56 of the genetic material.

41. The in vitro method of claim 23, wherein step (1) is preceded by the following step:
(1′) isolating genetic material of the HCMV from the subject.

42. The in vitro method of claim 23, wherein a positive finding under step (3) of claim 23 indicates a reduced therapeutic susceptibility of a subject infected by HCMV to a 3,4 dihydroquinazoline or a compound of Formula (X) and the administration of said 3,4 dihydroquinazoline or said compound of Formula (X) for use in a method of treatment of HCMV infection is not recommended; and wherein a negative finding under step (3) of claim 23 indicates no altered therapeutic susceptibility of a subject infected by HCMV to a 3,4 dihydroquinazoline or a compound of Formula (X) and the administration of said 3,4 dihydroquinazoline or said compound of Formula (X) for use in a method of treatment of HCMV infection is recommended.


SEQUENCE LISTING (22 sequences)

SEQ ID NO: 1 - protein, 850 aa, Human herpesvirus 5 (“Merlin”)
MEMNLLQKLCVVCSKCNEYAMELECLKYCDPNVLLAESTPFKRNAAAIVYLYRKIYPEVVAQNRTQSSLLTLYLEMLLKALHEDTALLDRALMAYSRQPDRAAFYRTVLRLDRCDRHHTVELQFTDNVRFSVSLATLNDIERFLCKMNYVYGILAPEAGLEVCAQLLELLRRLCGISPVARQEVYVEGTTCAQCYEELTIIPNQGRSLNKRLQGLLCNHIAVHRPSSQSDVNIQTVEQDLLDLTTRIPHLAGVLSALKSLFSSSSAYHSYIQEAEEALREYNLFTDIPERIYSLSDFTYWSRTSEVIVKRVGITIQQLNVYHQLCRALMNGISRHLYGEDVEDIFVLGEKALDGEERMFVGSVFAAPNRIIDLITSLSIQAFEDNPVFNKLHESNEMYTKIKHILEEIRRPLPDGTGGDGPEGEVIHLRGREAMSGTGTTLMTASNSSNSSTHSQRNNGGGGRARGGGKKAVGGGANGQDGDGSENGLRVRNCDEHEALDLVDARSRIHNVTREVNVRKRAYLQKVSEVGYGKVIRCIKTQERLTSKLIDVNLVGPLCLDFISKLMNGFLYRSQYHQDQDVVDVGNQFTYDEHLYVVNNLIHKSLPVESLPLLGQQIYELCNGPLFTHCTDRYPLSHNVDMAYACDNAGVLPHVKDDLVKCAEGTVYPSEWMVVKYMGFFNFSDCQDLNVLQKEMWMHVRELVLSVALYNETFGKQLSIACLRDELHPDRDVILTYNKEWPLLLRHEGSLYKSKDLYLLLYRHLSRPDESGDVPTAPVAKPSTLTAAAAVSGAFREPDRPWLPSPYPSSSTAGVSRRVRATRKRPRRASSLLDLARDEHGIQDLVPGSLR

SEQ ID NO: 2 - protein, 850 aa, Human herpesvirus 5 (AD169)
MEMNLLQKLCVVCSKCNEYAMELECLKYCDPNVLLAESTPFKRNAAAIVYLYRKIYPEVVAQNRTQSSLLTLYLEMLLKALHEDTALLDRALMAYSRQPDRAAFYRTVLRLDRCDRHHTVELQFTDNVRFSVSLATLNDIERFLCKMNYVYGILAPEAGLEVCAQLLELLRRLCGISPVARQEVYVEGTTCAQCYEELTIIPNQGRSLNKRLQGLLCNHIAVHRPSSQSDVNIQTVEQDLLDLTTRIPHLAGVLSALKSLFSSSSAYHSYIQEAEEALREYNLFTDIPERIYSLSDFTYWSRTSEVIVKRVGITIQQLNVYHQLCRALMNGISRHLYGEDVEDIFVLGEKALDGEERMFVGSVFAAPNRIIDLITSLSIQAFEDNPVFNKLHESNEMYTKIKHILEEIRRPLPDGTGGDGPEGEAIHLRGREAMSGTGTTLMTASNSSNSSTHSQRNNGGGGRARGGGKKVVGGGVNGQDGDGSENGLRVRNCDEHEALDLVDARSRIHNVTREVNVRKRAYLQKVSEVGYGKVIRCIKTQERLTSKLIDVNLVGPLCLDFISKLMNGFLYRSQYHQDQDVVDVGDQFTYDEHLYVVNNLIHKSLPVESLPLLGQQIYELCNGPLFTHCTDRYPLSHNVDMAYACDNAGVLPHVKDDLVKCAEGTVYPSEWMVVKYMGFFNFSDCQDLNVLQKEMWMHVRELVLSVALYNETFGKQLSIACLRDELHPDRDVILTYNKEWPLLLRHEGSLYKSKDLYLLLYRHLSRPDESGDVPTAPVAKPSTLTAAAAVSGVFREPDRPWLPSPYPSSSTAGVSRRVRATRKRPRRASSLLDLARDEHGIQDLVPGSLR

SEQ ID NO: 3 - DNA, 2553 bp, Human herpesvirus 5 (“Merlin”)
atggagatgaatttgttacagaaactatgcgtagtgtgttcgaaatgcaacgaatacgccatggagctggagtgtctaaagtactgcgatccgaacgtgttactggcggagtccacgccgttcaagagaaacgcggcggctatagtgtatctgtaccggaagatctacccggaggtggtggcgcagaatcgtacgcagagttcgctgctgactctctatctggagatgctgctgaaggcgctgcacgaggatacggctttgctggatcgggcgctgatggcctactcgcgccagccggaccgggcggccttctaccgtaccgtcctccgtttggatcgctgcgatcgccatcacaccgtggagctccagtttacggacaacgtccgtttcagcgtcagtctggccacactcaacgacatcgagcgcttcctgtgcaaaatgaactacgtgtacgggatcctggcgccggaggccggcctggaggtctgcgcgcagctgctggagctcctccgtcgcctatgcggcatctcgccggtggcgcgtcaggaagtgtacgtcgaagggacgacatgcgcccaatgctacgaggagctgaccatcatcccgaatcagggccgctcgctgaacaagcggctgcagggcttgctgtgcaaccatatagcggtccaccgtccgtcaagccagtccgatgtgaatatccagacggtggagcaggacctgctggacctgacaacgcgcatcccccacttggctggagtcctttcggccctcaaaagcctattctcttcttcatcggcctaccacagctacatccaggaggcggaggaggcgctgagggagtacaacctgtttacggatataccggaacgaatatattccttgtcggattttacctactggtcccgtacctcggaggttatcgtcaagcgggtgggcatcaccatccagcagctaaatgtgtatcaccagctgtgccgggcgctcatgaacggcatcagtcgccatctgtacggggaggacgtggaggacatcttcgtgctcggggaaaaggcgttggacggggaggagcgcatgttcgtggggtcggtctttgccgcccccaacaggatcatcgacctcatcacatccctcagcattcaagctttcgaggacaacccggtgttcaacaagctccacgaaagcaacgagatgtacaccaaaatcaagcatattctcgaggagattcgacgtccgctgcccgatggcacggggggcgacggccccgagggcgaggttattcacctgcgtggacgggaggcgatgtcggggacgggtacgactttgatgacggccagcaacagcagcaacagcagtactcacagtcagaggaataacggtggcggcggccgggcccgtggaggaggcaagaaagctgtagggggaggggcgaatgggcaggacggcgacggcagcgaaaacggcttacgggtgcgcaactgcgacgaacacgaagctctcgacttggtggacgcgcgttcccgcatccacaacgtcacccgcgaggtgaacgtgcgcaaacgcgcctacctgcagaaggtctcggaggtgggctatggcaaggtgatccgttgcatcaaaacgcaggagcgtctgactagcaaactcatcgatgtcaacctagtggggcccttgtgtctggactttatctctaagctcatgaatgggtttctataccgcagccaataccaccaggaccaagacgtggtggacgtggggaatcagttcacgtacgatgagcacctgtatgtggtcaataacctgatccacaagagtctgcctgtggaatccctcccgctactgggtcagcagatctacgagttgtgtaacgggcccctcttcacacactgtaccgatcgctatcccctctctcacaatgtggacatggcctatgcctgcgacaacgcgggcgtactaccccacgtcaaggacgatttggtcaaatgcgcggaaggtaccgtgtatcccagtgagtggatggtggtgaagtacatgggttttttcaatttttcggactgtcaggacctaaacgtgctgcagaaggagatgtggatgcacgtgcgggagctcgtgctctccgtcgcactatataatgaaactttcgggaaacaactctcgatcgcgtgcctgcgcgacgaactgcacccggacagagatgtgattctcacgtataacaaagagtggccgctgctgcttcgtcacgaaggaagtctttataagtccaaagatctatatctcctcctctacaggcatctgtccagaccggatgagagtggcgacgtgccaacagctcccgtggccaagccctccaccctgacggccgccgcggccgtctcgggtgccttcagggagccggaccgaccttggctgccaagtccgtacccctcctcctcgaccgcgggcgtttcccggagggtccgcgcaacacgcaagagaccacgacgcgcctcatcgctgctggatttggcccgcgacgaacatggaatccaggatctggtgcctggtagtctgcgttaa

SEQ ID NO: 4 - DNA, 2553 bp, Human herpesvirus 5 (AD169)
atggagatgaatttgttacagaaactatgcgtagtgtgttcgaaatgcaacgaatacgccatggagctggagtgtctaaagtactgcgatccgaacgtgttactggcggagtccacgccgttcaagagaaacgcggcggctatagtgtatctgtaccggaagatctacccggaggtggtggcgcagaatcgtacgcagagttcgttgctgacgctctacctggagatgctgctgaaggcgctgcacgaggatacggctttgctggatcgggcgctgatggcctactcgcgccagccggaccgggcggccttctaccgtaccgtcctccgtttggatcgctgcgatcgccatcacaccgtggagctccagtttacggacaacgtccgtttcagcgtcagtctggccacactcaacgacatcgagcgcttcctgtgcaaaatgaactacgtgtacgggatcctggcgccggaggccggcctggaggtctgcgcgcagttgctggagctcctccgtcgcctatgcggcatctcgccggtggcgcgtcaggaagtgtacgtcgaagggacgacatgcgcccaatgctacgaggagctgaccatcatcccgaatcagggccgctcgctgaacaagcggctgcagggcttgctgtgcaaccatatagcggtccaccgtccgtcaagccagtccgatgtgaatatccagacggtggagcaggacctgctggacctgacaacgcgcatcccccacttggctggagtcctttcggccctcaaaagcctattctcttcttcatcggcctaccacagctacatccaggaggcggaggaggcgctgagggagtacaacctgtttacggatataccggaacgaatatattccttgtcggattttacctactggtcccgtacctcggaggttatcgtcaagcgggtgggcatcaccatccagcagctaaatgtgtatcaccagctgtgccgggcgctcatgaacggcatcagtcgccatctgtacggggaggacgtggaggacatcttcgtgctcggggaaaaggcgttggacggggaggagcgcatgttcgtggggtcggtctttgccgcccccaacaggatcatcgacctcatcacatccctcagcattcaagctttcgaggacaacccggtgttcaacaagctccacgaaagcaacgagatgtacaccaaaatcaagcatattctcgaggagattcgacgtccgctgccagatggcacggggggcgacggccccgagggcgaggctattcacctgcgtggacgggaggcgatgtcggggacgggtacgactttgatgacggccagcaacagcagcaacagcagtactcacagtcagaggaataacggtggcggcggccgggctcgtggaggaggcaagaaagtggtagggggaggggtgaatgggcaggacggcgacggcagcgagaacggcttacgggtgcgcaactgcgacgaacacgaggctctcgacttggtggacgcgcgctcccgcatccacaacgtcactcgcgaggtgaacgtgcgcaaacgcgcctacctgcagaaggtctcggaggtgggctatggcaaggtgatccgttgcatcaaaacgcaggagcgtctgactagcaagctcatcgatgtcaatctagtggggcccttgtgtctggactttatctctaagctcatgaatgggtttctgtaccgcagccaataccaccaggaccaagacgtggtggacgtgggtgatcagttcacctacgatgagcacctgtatgtggtcaataacctgatccacaagagtctgcctgtggaatccctcccgctactgggtcagcagatctacgagttgtgtaacgggcccctcttcacccactgcaccgatcgttatcccctctctcacaatgtggacatggcctatgcctgcgacaacgcgggcgtactaccccacgtcaaggacgatttggtcaaatgcgcggaaggtaccgtgtatcccagtgagtggatggtggtgaagtatatgggttttttcaatttttcggactgtcaggacctaaacgtgctgcagaaggagatgtggatgcacgtgcgggagctcgtgctctccgtcgcactatataatgaaactttcgggaaacaactctcgatcgcgtgcctgcgcgacgaactgcacccggacagagatgtgattctcacgtataacaaagagtggccgctgctgcttcgtcacgaaggaagtctttataagtccaaagatctatatctcctcctctacaggcatctgtccagaccggatgagagtggcgacgtgccaacagctcccgtggccaagccctccaccctgacggccgccgcggccgtctcgggtgtcttcagggagccggaccgaccttggctgccaagtccgtatccctcctcctcgactgcgggtgtttcccggagggtccgcgcgacacgcaagagaccacgacgcgcctcatcgctgctggatttggcccgcgacgaacatggaatccaggatctggtgcctggtagtctgcgttaa

SEQ ID NO: 5 - DNA, 20 bp, Artificial sequence
  PCR Primer
acgcggcggctatagtgtat

SEQ ID NO: 6 - DNA, 20 bp, Artificial sequence
  PCR Primer
gcatccacatctccttctgc

SEQ ID NO: 7 - DNA, 20 bp, Artificial sequence
  PCR Primer
agctgaccatcatcccgaat

SEQ ID NO: 8 - DNA, 22 bp, Artificial sequence
  PCR Primer
ccacattgtgagagaggggata

SEQ ID NO: 9 - DNA, 20 bp, Artificial sequence
  PCR Primer
gtccacgcaggtgaatagcc

SEQ ID NO: 10 - DNA, 20 bp, Artificial sequence
  PCR Primer
aaaccgcagcacccagacag

SEQ ID NO: 11 - DNA, 20 bp, Artificial sequence
  PCR Primer
tacggctttgctggatcggg

SEQ ID NO: 12 - DNA, 20 bp, Artificial sequence
  PCR Primer
cgcgtcaggaagtgtacgtc

SEQ ID NO: 13 - DNA, 22 bp, Artificial sequence
  PCR Primer
tgtcggattttacctactggtc

SEQ ID NO: 14 - DNA, 23 bp, Artificial sequence
  PCR Primer
gcaacgagatgtacaccaaaatc

SEQ ID NO: 15 - DNA, 20 bp, Artificial sequence
  PCR Primer
tgctgttgctggccgtcatc

SEQ ID NO: 16 - DNA, 20 bp, Artificial sequence
  PCR Primer
gaggtgggctatggcaaggt

SEQ ID NO: 17 - DNA, 21 bp, Artificial sequence
  PCR Primer
tgtggtcaataacctgatcca

SEQ ID NO: 18 - DNA, 20 bp, Artificial sequence
  PCR Primer
actgcaccgatcgttatccc

SEQ ID NO: 19 - DNA, 20 bp, Artificial sequence
  PCR Primer
agatgtggatgcacgtgcgg

SEQ ID NO: 20 - DNA, 22 bp, Artificial sequence
  PCR Primer
ggagatatagatctttggactt

SEQ ID NO: 21 - DNA, 20 bp, Artificial sequence
  PCR Primer
tctcgggtgtcttcagggag

SEQ ID NO: 22 - DNA, 20 bp, Artificial sequence
  PCR Primer
agcggcccgcgagttatttg